SEQUENCE LISTING (7 sequences)

SEQ ID NO: 1 - protein, 72 aa, Artificial Sequence
  Synthetic
TENEIRKLRKLLRIAMLLLVFLLIATVVSLWTSKTDDDPSAQSEQLVAMSLMLIAASLLIIAISKLLKSRNG

SEQ ID NO: 2 - protein, 100 aa, Artificial Sequence
  Synthetic
SAEELLRRSREYLKKVKEEQERKAKEFQELLKELSERSEELIRELEEKGAASEAELARMKQQHMTAYLEAQLTAWEIESKSKIALLELQQNQLNLELRHI

SEQ ID NO: 3 - protein, 100 aa, Artificial Sequence
  Synthetic
  MISC_FEATURE (1)..(1): optionally absent
  MISC_FEATURE (98)..(100): optionally absent
SAEELLRRSREYLKKVALIQLVIAFVFLILLILLSWRSEELIRELEEKGAASEAELARMKQQHMTAYLQAALTAWEIISKSVIALLLLQQNQLNLELRHI

SEQ ID NO: 4 - protein, 203 aa, Artificial Sequence
  Synthetic
SAEELLRRSREYLKKVALIQLVIAFVFLILLILLSWRSEELIRELEEKGAASEAELARMKQQHMTAYLQAALTAWEIISKSVIALLLLQQNQLNLELNTDTDKNVAEELLRRSREYLKKVALIQLVIAFVFLILLILLSWRSEELIRELEEKGAASEAELARMKQQHMTAYLQAALTAWEIISKSVIALLLLQQNQLNLELRH

SEQ ID NO: 5 - protein, 72 aa, Artificial Sequence
  Synthetic
TEDEIRKLRKLLEEAEKKLKKLEDKTRRSEEISKTDDDPKAQSLQLIAESLMLIAESLLIIAISLLLSSRNG

SEQ ID NO: 6 - protein, 72 aa, Artificial Sequence
  Synthetic
  MISC_FEATURE (3)..(3): X is D or N
  MISC_FEATURE (47)..(47): X is V or I
TEXEIRKLRKLLREAELLLKKLEDATRRSEEISKTDDDPKAQSEQLXAESLMLIAASLLIIAISLLLKSRNG

SEQ ID NO: 7 - protein, 100 aa, Artificial Sequence
  Synthetic
  MISC_FEATURE (69)..(69): X is Q or E
SAEELLRRSREYLKKVAEEQERIAKEFQELLIELSERSEELIRELEEKGAASEAELARMKQQHMTAYLXAALTAWEIESKSKIALLELQQNQLNLELRHI

(19) United States
(12) Patent Application Publication
(10) Pub. No.: US 2023/0192773 A1
(43) Pub. Date: Jun. 22, 2023

(54) DE NOVO DESIGNED ALPHA-HELICAL PROTEIN CHANNELS

(71) Applicant: University of Washington, Seattle, WA (US)

(72) Inventors: Chunfu XU, Seattle, WA (US); Peilong LU, Seattle, WA (US); Tamer GAMAL EL-DIN, Seattle, WA (US); William A. CATTERALL, Seattle, WA (US); David BAKER, Seattle, WA (US)

(21) Appl. No.: 17/907,302

(22) PCT Filed: Apr. 28, 2021
(86) PCT No.: PCT/US2021/029533

§ 371 Date: Sep. 26, 2022

Related U.S. Application Data
(60) Provisional application No. 63/017,810, filed on Apr. 30, 2020.

(51) Int. Cl. C07K 14/00; C07K 14/705

(52) U.S. Cl. CPC C07K 14/001; C07K 14/705; C07K 2319/03; C12Q 1/6869

(57) ABSTRACT

Disclosed herein are polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NOS: 1-4, oligomers thereof, and uses thereof.

### CROSS REFERENCE

This application claims priority to U.S. Provisional Patent Application Ser. No. 63/017810 filed Apr. 30, 2020, incorporated by reference herein in its entirety.

### FEDERAL FUNDING STATEMENT

This invention was made with government support under Grant No. FA9550-18-1-0297, awarded by the Air Force Office of Scientific Research. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Apr. 8, 2021, having the file name “20-596-WO_SeqList_ST25.txt” and is 8 kb in size.

### BACKGROUND

Protein pores play key roles in fundamental biological processes and biotechnological applications such as DNA nanopore sequencing, and hence the design of pore-containing proteins is of considerable scientific and biotechnological interest.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NOS: 1-4. In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 1, wherein the polypeptide is identical to the polypeptide of SEQ ID NO:1 at least at 1, 2, 3, 4, 5, 6, 7, 8, 9, 10, 11, 12, 13, 14, 15, 16, 17, 18, 19, 20, 21, 22, or more of residues 3, 13-14, 16-18, 20, 21, 23-25, 27-28, 30-32, 40, 44, 47, 49, 56, 65, and 68 in SEQ ID NO:1. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 1, wherein changes in the polypeptide amino acid sequence relative to SEQ ID NO:1 comprise:

(a) changes in 1, 2, 3, 4, 5, 6, 7, 8, 9, 10, 11, 12, 13, 14, 15, 16, 17, 18, 19, 20, 21, or more of residues 1-4, 6-7, 9-10, 32-40, 42, and 70-72 to other polar amino acids;

(b) changes in 1, 2, 3, 4, 5, 6, 7, 8, 9, 10, 11, 12, 13, or more of residues 14, 16-18, 20-21, 23-25, 27-28, 30, 49, 56, and 63 to other hydrophobic amino acids; and/or

(c) a change at residue 44.

In a further embodiment, the polypeptide is identical to SEQ ID NO:1 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, or all of residues 5, 8, 11-12, 15, 19, 22, 26, 29, 41, 43, 45-48, 50, 51-55, 57-62, 64, and 67-69 in SEQ ID NO:1. In one embodiment, the polypeptide is identical to SEQ ID NO:1 at 1, 2, or all 3 of residues 13, 31, and 65 in SEQ ID NO:1.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 2, wherein changes in the polypeptide amino acid sequence relative to SEQ ID NO:2 comprise changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14-15, 17-19, 21-26, 28-29, 32-33, 36-37, 39-40, 43-44, 46-55, 58, 60, 67, 71, 78, 82, 96, and 98-100 to other polar amino acids. In one embodiment, the polypeptide is identical to SEQ ID NO:2 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:2. In another embodiment, the polypeptide is identical to SEQ ID NO:2 at least at 1, 2, 3, 4, 5, 6, 7, 8, 9, or more of residues 62, 66, 69, 73, 76, 80, 83, 87, 91, and 94 in SEQ ID NO:2.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 3, wherein changes in the polypeptide amino acid sequence relative to SEQ ID NO:3 comprise

(a) changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14, 37, 39-40, 43-44, 46-55, 58, 60, 96, and 98-100 to other polar amino acids; and/or

(b) changes in 1, 2, 3, 4, 5, 6, 7, 8, 9, 10, 11, 12, 13, 14, 15, or more of residues 17-19, 21-26, 28-29, 32-33, 71, 78, and 82 to other hydrophobic amino acids.

In another embodiment, the polypeptide is identical to SEQ ID NO:3 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:3. In a further embodiment, the polypeptide is identical to SEQ ID NO:3 at 1, 2, or all 3 of residues 15, 36, and 66 in SEQ ID NO:3. In one embodiment, the polypeptide is identical to SEQ ID NO:3 at least at 1, 2, 3, 4, 5, 6, 7, 8, 9, or more of residues 62, 66, 69, 73, 76, 80, 83, 87, 91, and 94 in SEQ ID NO:3. In another embodiment, all optional amino acid residue positions in the polypeptide relative to SEQ ID NO:3 are present.

In one embodiment, the polypeptide is of the formula X1-Z—X2, wherein X1 and X2 independently comprise the polypeptide of any embodiment or combination of embodiments of the polypeptides disclosed herein, and Z comprises an amino acid linker. In another embodiment, the polypeptide is of the formula X1-Z—X2, wherein X1 and X2 independently comprise any embodiment or combination of embodiments of the polypeptides disclosed herein, and Z comprises an amino acid linker. In a further embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4.

In another embodiment, the disclosure provides polypeptide oligomers comprising 2, 3, 4, 5, 6, or more copies of the polypeptide of any embodiment or combination of embodiments of the polypeptides disclosed herein. In one embodiment, the oligomer comprises a homo-hexamer of the polypeptide of any embodiment or combination of embodiments of the polypeptides disclosed herein.

In other aspects, the disclosure provides nucleic acids encoding the polypeptide of any embodiment or combination of embodiments of the polypeptides disclosed herein, expression vector comprising a nucleic acid of the disclosure operatively linked to a suitable control sequence, and host cells comprising the nucleic acid, expression vector, polypeptide, or polypeptide oligomer of any embodiment or combination of embodiments disclosed herein. In one embodiment of host cells of the disclosure, the polypeptide oligomer forms a transmembrane pore in the host cell. In another aspect, the disclosure provides liposomes comprising the polypeptide oligomer of any embodiment herein, wherein the polypeptide oligomer forms a transmembrane channel in the liposome. In a further aspect, the disclosure provides uses or methods of use of the polypeptides, polypeptide oligomers, nucleic acids, vectors, host cells, and liposomes described herein for any suitable purpose, including but not limited to selective transmembrane ion conductance, DNA nanopore sequencing, generating membranes with selective permeabilities, sensing molecules in the environment, and controlling cellular behavior.

### DESCRIPTION OF THE FIGURES

FIG. 1(a-e). The biophysical characterizations and ion conductivity of the 12-helix TMHC6 transmembrane channel. ((a) TMHC6 channel model. The permeation path, calculated by HOLE20, is illustrated in the left panel. Constriction sites along the channel are the E-ring (E44), the K-rings (K65, K68), and two intervening L-rings (L51, L58). (right) The radius of the pore along the permeation path. (b) CD spectra and temperature melt (inset). No apparent unfolding transition is observed up to 95° C. Re25° C. spectrum is taken when the sample cools back to 25° C. after the thermal melt scan. (c) AUC sedimentation-equilibrium curves at three different rotor speeds for TMHC6 yield a measured molecular weight of ˜57 kDa consistent with the designed hexamer. “MW (D)” and “MW (E)” indicate the molecular weight of the oligomer design and that calculated from the experiment, respectively. (d) Conductivity in whole-cell patch clamp experiment on insect cells expressing TMHC6. The channel blocker Gd3+ diminished ion conductance to that of untransfected cells. (e) TMHC6 has considerably higher conductance for K+ than Na+, Cs+, CH3NH3+ and Ba2+. The error bars indicate the standard error of measurement (s.e.m.).

FIG. 2(a-d). Blocking of the ion conductance of TMHC6 with site-directed mutagenesis and chemical biology. (a) The extracellular ring of six Glu44 residues (E-ring, shown as sticks) is a likely site for cation entry. (b) Cd2+ blocking of the potassium conductance of TMHC6 and the E44H mutant. 3-fold higher Cd2+ concentrations were required to block the E44H mutant compared to the wildtype, likely because of the reduced electrostatic attraction. The error bars indicate the s.e.m. (c,d) Blocking of the conductance of the TMHC6 E44C mutant using cysteine reactive reagents. Negatively charged MTSES, positively charged MTSET, and hydrophobic MTS-TBAE all completely blocked the ion conductance of the E44C mutant within a few minutes under voltage clamp control, while they had no effect on TMCH6 itself in control experiments. The error bars indicate the s.e.m.

FIG. 3(a-d). The X-ray crystal structure of water-soluble WSHC8 and the design model of the 16-helix TMH4C4 transmembrane channel. (a) Superposition of backbones of the crystal structure and the design model of WSHC8. The C-a RMSD is 2.51 Å. The larger deviation for the octamer is caused by the slight tilting of the hairpin monomers along the superhelical axis of the complex. (b) Superposition of the crystal structure and the design model of the WSHC8 monomer. The C-a RMSD is 0.97 Å. (c) The cross section of the WSHC8 channel. (d) TMH4C4 model with 16 transmembrane helices.

FIG. 4(a-j). Design and solution characterization of water-soluble pores. Top row, 12 helix design WSHC6; bottom row, 16 helix design WSHC8. (a & f) Design models. (b & g) Energy versus RMSD plots generated from Rosetta™ “fold-and-dock” structure prediction calculations. The predicted structures converge on the design models with RMSD values less than 2.0 Å. Structures in the alternative energy minima at large RMSD positions also recapitulate the design models but with chain identities in the RMSD calculations reversed. (c & h) Wavelength-scan and temperature-scan (insets) CD spectra. WSHC6 does not melt up to 95° C., while WSHC8 has a melting temperature of 85° C. The overlap of the pre- and post-annealing CD spectra indicates that the thermal denaturation is reversible. (d & i) Representative analytical ultracentrifugation sedimentation-equilibrium curves at three different rotor speeds for WSHC6 and WSHC8, 0.2 OD230 and 0.3 OD230 in PBS (pH 7.4), respectively. The determined oligomeric states match those of the design models. (e & j) Small-angle X-ray Scattering (SAXS) characterization. The experimental scattering profiles are similar to scattering profiles computed from the design models.

FIG. 5(a-b). Representative gel filtration chromatography and SDS-PAGE of WSHC6 and WSHC8. (A) Gel filtration curves for WSHC6 and WSHC8 with 6x histidine tags (solid lines) and after cleaving off histidine tags (dotted lines). (B) SDS-PAGE of WSHC6 (lane 1) and WSHC8 (lane 2) after purification with histidine tags removed.

FIG. 6(a-c). Molecular weight determinations using SEC-MALS. (A) The determined MW for WSHC6 was 52.9 kDa, consistent with the formation of hexamers in solution. (B) WSHC8 had a determined MW of 94.9 kDa suggesting the formation of octameric assemblies. (C) Another octameric design was determined to form the expected oligomerization state.

FIG. 7(a-d). Additional analytical ultracentrifugation sedimentation-equilibrium curves at different protein concentrations and rotor speeds. (A-B) WSHC6. (C-D) WSHC8.

FIG. 8(a-b). Comparisons of the transmembrane protein sequences to water-soluble protein sequences. Hydrophobic TMs are indicated above the sequences. (A) Sequence alignment of TMHC6 (SEQ ID NO: 1) with WSHC6 (SEQ ID NO: 5) (consensus is SEQ ID NO: 6). (B) Sequence alignment of TMHC8 (SEQ ID NO: 3) with WSHC8 (SEQ ID NO: 2) (consensus is SEQ ID NO: 7).

FIG. 9(a-b). Pore-lining residues in WSHC6 and TMHC6. (A) Overlay of the crystal structure and the design model of WSHC6. The pore of this water-soluble protein is lined with alternating leucine and isoleucine residues. (B) The TMHC6 pore is lined with hydrophobic leucine and isoleucine residues and polar rings at the extracellular and intracellular sides, E44 ring and K65 ring.

FIG. 10(a-b). Purification of TMHC6 and variants. (A) Representative gel filtration chromatography and SDS-PAGE of TMHC6 and E44F mutant. All the two constructs could be expressed in E. coli and purified to homogeneity. (B) Gel filtration chromatography and SDS-PAGE of TMHC6 E44C mutant, treated or untreated with MTSES reagent.

FIG. 11. Analytical ultracentrifugation sedimentation-equilibrium curves of TMHC6. AUC sedimentation-equilibrium curves are shown at three different rotor speeds for two different concentrations for TMHC6. Together with the data in FIG. 3, three data sets are well fit globally as a single ideal species in solution corresponding to the molecular weight of a hexamer. ‘MW (D)’ refers to the molecular weight of the oligomer design and ‘MW (E)’ refers to the molecular weight determined in the experiment. FIG. 12. Negative stain EM for TMHC6 in amphipols. Protein particles on the EM grid showed round shape and size consistent with the soluble protein crystal structure and the design model (scale bar at the bottom left, 100 nm). Inset: close-up view of representative particles; each side of the particle frames represent 12.8 nm.

FIG. 13. Disrupting mutation in the pore entrance reduces the current. The E44F single mutant reduced the K+current to half and one third of that for TMHC6. TMHC2, a previously designed transmembrane protein without a pore, does not conduct ions across the membrane. The error bars indicate the s.e.m.

FIG. 14. Expression of TMHC6 and mutants in insect cells for the whole-cell patch clamp experiment. The same amount of cells were loaded into the gel and the expression levels for two variants were examined by western blot. The E44F mutant had similar, if not higher, expression level as TMHC6. The E44C mutant expressed at a slightly lower level compared to TMHC6.

FIG. 15(a-f). Comparison of first round and second round octameric designs. (A) A design model from the first round of design. (B) The monomers of the first round designs have 70 amino acids. The intersubunit interfaces consist of primarily hydrophobic residues and minimal hydrogen bonding interactions. (C) The design model of WSHC8. (D) The monomers of WSHC8 have 100 residues. The increased intersubunit interface allows the placement of extensive hydrogen bonding networks without compromising hydrophobic packing. Example hydrogen bonding networks in the first round of design (E) and in WSHC8 (F).

FIG. 16(a-b). Pore-lining residues in WSHC8 and TMH4C4. (A) Overlay of the crystal structure and the design model of WSHC8. Helices are more tilted in the crystal structure than in the design model. The lumen of this larger pore is freely water-accessible, so the residues inside the pore are all polar. Shown in the figure are three representative layers of the pore-lining residues in the crystal structure, Glu69 ring, Lys80 ring, and Glu87. The missing heavy atoms of these flexible residues are built using Rosetta™ with backbone constraints. (B) Overlay of the cryo-EM structure and the design model of TMH4C4 which is based on the crystal structure of WSHC8. Shown on the right are three pore-lining layers in the cryo-EM structure corresponding to the three layers in (A). Glu69 and Glu87 are replaced with glutamine and leucine, respectively, in the transmembrane pore to better comply with the “positive-inside” rule.

FIG. 17(a-b). Purification of designer transmembrane pores. (A) Representative gel filtration chromatography and SDS-PAGE of TMHC8. The protein was purified in the buffer containing 0.2% (weight/volume) n-decyl-β-D-maltopyranoside (DM, Anatrace). (B)

Representative gel filtration chromatography and SDS-PAGE of TMH4C4. TMH4C4 protein was purified in the buffer containing 0.2% (weight/volume) n-decyl-β-D-maltopyranoside (DM, Anatrace).

FIG. 18(a-b). CD spectra and thermal stability of 16-helix transmembrane pores. (A) CD spectra of TMHC8 at different temperatures. An unfolding transition is observed at ˜75° C. (B) CD spectra of TMH4C4. The protein is thermally stable up until 95° C.

FIG. 19(a-b). Analytical ultracentrifugation sedimentation-equilibrium curves of 16-helix transmembrane nanopores. Analytical ultracentrifugation sedimentation-equilibrium curves of TMHC8 (panel A) and TMH4C4 (panel B). AUC sedimentation-equilibrium curves are shown at three different rotor speeds for different concentrations for TMHC8 and TMH4C4. For TMHC8, three data sets are well fit globally as a single ideal species in solution corresponding to the molecular weight of 98.9 kDa, which is in between the MWs of a heptamer and an octamer. For TMH4C4 tetramer, two data sets are globally fitted as a single species with a molecular weight of 98.1 kDa, which is very close to the MW of a tetramer. ‘MW (D)’ refers to the molecular weight of the oligomer design and ‘MW (E)’ refers to the molecular weight determined in the experiment.

FIG. 20. The narrowest dimension of the head group of Alexa™ Fluor 488-biocytin is approximately 12 Å. The Van der Waals radius (red) and the distance (blue) between two nitrogen atoms are labeled. The 10 Å channel of TMH4C4, but not the TMHC6 channel with the 3.3 Å constriction calculated from the design model, would be expected to allow passage of the dye allowing for thermal fluctuations of the side chains and backbone.

### DETAILED DESCRIPTION

As used herein, the singular forms “a”, “an” and “the” include plural referents unless the context clearly dictates otherwise. “And” as used herein is interchangeably used with “or” unless expressly stated otherwise.

All embodiments of any aspect of the disclosure and appendices can be used in combination, unless the context clearly dictates otherwise.

Unless the context clearly requires otherwise, throughout the description, appendix, and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As referred to herein, polar amino acid residues are Gly (G), Ser (S), Thr (T), Cys (C), Tyr (Y), Asn (N), Gln (Q); Asp (D), Glu (E); Lys (K), Arg (R), and His (H). As referred to herein, hydrophobic residues are Ala (A), Val (V), Leu (L), Ile (I), Pro (P), Phe (F), Trp (W), and Met (M).

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NOS: 1-4. As described in detail herein, the inventors describe the computational design of protein channels or pores formed by two concentric rings of α-helices that are stable and mono-disperse in both water-soluble and membrane-embedded forms. The protein channels or pores are comprised of polypeptide subunits as claimed herein.

TMHC6
(SEQ ID NO: 1)
TENEIRKLRKLLRIAMLLLVFLLIATVVSL
WTSKTDDDPSAQSEQLVAMSLMLIAASLLI
IAISKLLKSRNG
72 AAs

As described in the examples, the polypeptides can tolerate significant substitutions, as described in detail below. For example with respect to SEQ ID NO:1 (TMHC6):

Residues 1-4, 6-7, 9-10, 32-40, 42, and 70-72: Exposed surface residues that can be mutated to other polar residues

Residues 14, 16-18, 20-21, 23-25, 27-28, 30, 49, 56, 63: Exposed lipid interacting residues in the TM domains that can be mutated to other hydrophobic residues.

Residues 5, 8, 11-12, 15, 19, 22, 26, 29, 41, 43, 45-48, 50, 51-55, 57-62, 64, 67-69: Core residues buried inside the protein; useful to hold the hexameric topology / pore formation.

Residues 13-31; 44-65: Transmembrane (TM) domains

Residues 13, 31, and 65: Useful residues to set the orientation of the transmembrane protein in the lipid bilayer.

Residue 44: Ion selectivity filter. This residue provides K+ion selectivity; substitution of this residue can be used to modify ion selectivity or eliminate ion selectivity altogether.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 1, wherein the polypeptide is identical to the polypeptide of SEQ ID NO:1 at least at 1, 2, 3, 4, 5, 6, 7, 8, 9, 10, 11, 12, 13, 14, 15, 16, 17, 18, 19, 20, 21, 22, or more of residues 3, 13-14, 16-18, 20, 21, 23-25, 27-28, 30-32, 40, 44, 47, 49, 56, 65, and 68 in SEQ ID NO:1. These residues are highlighted in bold in SEQ ID NO:1.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 1, wherein changes in the polypeptide amino acid sequence relative to SEQ ID NO:1 comprise:

(a) changes in 1, 2, 3, 4, 5, 6, 7, 8, 9, 10, 11, 12, 13, 14, 15, 16, 17, 18, 19, 20, 21, or more of residues 1-4, 6-7, 9-10, 32-40, 42, and 70-72 to other polar amino acids;

(b) changes in 1, 2, 3, 4, 5, 6, 7, 8, 9, 10, 11, 12, 13, or more of residues 14, 16-18, 20-21, 23-25, 27-28, 30, 49, 56, and 63 to other hydrophobic amino acids; and/or

(c) a change at residue 44.

In a further embodiment, the polypeptide is identical to SEQ ID NO:1 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, or all of residues 5, 8, 11-12, 15, 19, 22, 26, 29, 41, 43, 45-48, 50, 51-55, 57-62, 64, and 67-69 in SEQ ID NO:1. These are the core residues of TMHC6.

In a further embodiment, the polypeptide is identical to SEQ ID NO:1 at 1, 2, or all 3 of residues 13, 31, and 65 in SEQ ID NO:1.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NOS: 2-4. In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 2, wherein changes in the polypeptide amino acid sequence relative to SEQ ID NO:2 comprise changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14-15, 17-19, 21-26, 28-29, 32-33, 36-37, 39-40, 43-44, 46-55, 58, 60, 67, 71, 78, 82, 96, and 98-100 to other polar amino acids.

WSHC8
(SEQ ID NO: 2)
SAEELLRRSREYLKKVKEEQERKAKEFQELLK
ELSERSEELIRELEEKGAASEAELARMKQQHM
TAYLEAQLTAWEIESKSKIALLELQQNQLNLE
LRHI
100 amino acids

As described herein with respect to SEQ ID NO:2 (WSHC8):

Residues 1-4, 7-8, 10-12, 14-15, 17-19, 21-26, 28-29, 32-33, 36-37, 39-40, 43-44, 46-55, 58, 60, 67, 71, 78, 82, 96, 98-100: exposed surface residues; can be mutated to other polar residues.

Residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, 97: core residues buried inside the protein; useful residues to hold the hexameric topology/pore formation.

Residues 62, 66, 69, 73, 76, 80, 83, 87, 91, 94: pore-facing residues; too many hydrophobic residues may collapse the pore.

In one embodiment, the polypeptide is identical to SEQ ID NO:2 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:2. These are the core residues.

In another embodiment, the polypeptide is identical to SEQ ID NO:2 at least at 1, 2, 3, 4, 5, 6, 7, 8, 9, or more of residues 62, 66, 69, 73, 76, 80, 83, 87, 91, and 94 in SEQ ID NO:2. These are the pore-facing residues.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 3, wherein changes in the polypeptide amino acid sequence relative to SEQ ID NO:3 comprise

(a) changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14, 37, 39-40, 43-44, 46-55, 58, 60, 96, and 98-100 to other polar amino acids; and/or

(b) changes in 1, 2, 3, 4, 5, 6, 7, 8, 9, 10, 11, 12, 13, 14, 15, or more of residues 17-19, 21-26, 28-29, 32-33, 71, 78, and 82 to other hydrophobic amino acids; and

wherein residues in parentheses are optional.

TMHC8
(SEQ ID NO: 3)
(S)AEELLRRSREYLKKVALIQLVIAFVFLI
LLILLSWRSEELIRELEEKGAASEAELARMK
QQHMTAYLQAALTAWEIISKSVIALLLLQON
QLNLEL (RHI)
100 amino acids

As described herein with respect to SEQ ID NO:3 (TMHC8):

Residues 1-4, 7-8, 10-12, 14, 37, 39-40, 43-44, 46-55, 58, 60, 96, 98-100: exposed surface residues; can be mutated to other polar residues.

Residues 17-19, 21-26, 28-29, 32-33, 71, 78, 82: exposed lipid interacting residues in the TM domains; can be mutated to other hydrophobic residues.

Residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, 97: core residues buried inside the protein; useful residues to hold the hexameric topology / pore formation.

Residues 62, 66, 69, 73, 76, 80, 83, 87, 91, 94: pore-facing residues; too many hydrophobic residues may collapse the pore.

Residues 16-35 and residues 68-87: TM domains.

Residues 15, 36, 66: useful residues to set the orientation of the transmembrane protein in the lipid bilayer.

In one embodiment, the polypeptide is identical to SEQ ID NO:3 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:3. These are the core residues.

In another embodiment, the polypeptide is identical to SEQ ID NO:3 at 1, 2, or all 3 of residues 15, 36, and 66 in SEQ ID NO:3.

In a further embodiment, the polypeptide is identical to SEQ ID NO:3 at least at 1, 2, 3, 4, 5, 6, 7, 8, 9, or more of residues 62, 66, 69, 73, 76, 80, 83, 87, 91, and 94 in SEQ ID NO:3. These are the pore-facing residues.

In one embodiment, all optional amino acid residue positions in the polypeptide relative to SEQ ID NO:3 are present. In other embodiments, one or more of the optional amino acid residues are absent. In one embodiment, residue 1 can be S or V. In another embodiment, the C-terminal optional residues (98-100) may RHI, RH, R, or absent, or may be absent or may be substituted with other amino acids.

In another embodiment, the polypeptide has the formula X1-Z—X2, wherein X1 and X2 independently comprise the polypeptide of any embodiment or embodiments described above related to SEQ ID NOS:1-3, and Z comprises an amino acid linker. Thus, in various embodiments X1 may comprise any embodiment disclosed herein where SEQ ID NO:1 is the reference sequence, where SEQ ID NO:2 is the reference sequence, or where SEQ ID NO:3 is the reference sequence, and X3 may independently comprise any embodiment disclosed herein where SEQ ID NO:1 is the reference sequence, where SEQ ID NO:2 is the reference sequence, or where SEQ ID NO:3 is the reference sequence. Any suitable linker may be used, including but not limited to those described herein. In certain embodiments, the linker comprises a flexible linker of at least 4 amino acids in length.

In one embodiment, X1 and X3 independently comprise any embodiment disclosed herein where SEQ ID NO:3 is the reference sequence. In these embodiments, X1 and X3 may comprise the same amino acid sequence or may differ. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4.

TMH4C4
(SEQ ID NO: 4)
SAEELLRRSREYLKKVALIQLVIAFVFLILL
ILLSWRSEELIRELEEKGAASEAELARMKQQ
HMTAYLQAALTAWEIISKSVIALLLLQQNQL
NLELNTDTDKNVAEELLRRSREYLKKVALIQ
LVIAFVFLILLILLSWRSEELIRELEEKGAA
SEAELARMKQQHMTAYLQAALTAWEIISKSV
IALLLLQQNQLNLELRH

In another embodiment, the disclosure provides polypeptide oligomers comprising 2, 3, 4, 5, 6, or more copies of the polypeptide of any embodiment or combination of embodiments disclosed herein. The polypeptide oligomers can function, for example, as protein channels or pores in water-soluble or membrane-embedded forms, as discussed in detail in the examples that follow.

In one embodiment, the polypeptide copies in the oligomer are identical, and the oligomer may comprise a homo-oligomer, including but not limited to a homo-dimer, homo-trimer, homo-tetramer, homo-pentamer, or homo-hexamer. In one specific embodiment, the oligomer comprises a homo-hexamer.

As used throughout the present application, the term “polypeptide” is used in its broadest sense to refer to a sequence of subunit D- or L-amino acids, including canonical and non-canonical amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides disclosed herein; these additional residues are not included in determining the percent identity of the polypeptides of the invention relative to the reference polypeptide.

In another aspect the disclosure provides nucleic acids encoding the polypeptides of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), polypeptide, or polypeptide oligomer of any embodiment or combination of embodiments disclosed herein, wherein the host cells can be either prokaryotic or eukaryotic. For example, the cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In one embodiment, the polypeptide oligomer forms a transmembrane pore in the host cell as described above and as detailed in the examples. In one such embodiment, the host cell is a eukaryotic cell.

In another aspect, the disclosure provides liposomes comprising the polypeptide oligomer of any embodiment herein, wherein the polypeptide oligomer forms a transmembrane channel in the liposome, as described in detail in the examples that follow. As used herein, a liposome is any spherical vesicle having at least one lipid bilayer.

In another aspect, the disclosure provides methods for use of the polypeptides, polypeptide oligomers, nucleic acids, vectors, host cells, and liposomes described herein for any suitable purpose, including but not limited to selective transmembrane ion conductance,

DNA nanopore sequencing, generating membranes with selective permeabilities, sensing molecules in the environment, and controlling cellular behavior.

### EXAMPLES

Protein pores play key roles in fundamental biological processes and biotechnological applications such as DNA nanopore sequencing, and hence the design of pore-containing proteins is of considerable scientific and biotechnological interest. The de novo design of stable, well-defined transmembrane protein pores capable of conducting ions selectively or large enough to allow passage of small-molecule fluorophores remains an outstanding challenge. Here, we report the computational design of protein pores formed by two concentric rings of α-helices that are stable and mono-disperse in both water-soluble and membrane-embedded forms. Crystal structures of the water-soluble forms of a 12 helical pore with an inner diameter at the narrowest constriction of 4 Å, and of a 16 helical pore with a constriction diameter of 10 Å, are close to the computational design models (0.89 and 2.51 Å C-α RMSD respectively). Patch clamp electrophysiology experiments show that the transmembrane form of the 12-helix pore expressed in insect cells allows passage of ions across the membrane with high selectivity for potassium over sodium, which is blocked by specific chemical modification at the pore entrance. The transmembrane form of the 16-helix pore, but not the 12-helix pore, allows passage of biotinylated Alexa™ Fluor 488 when incorporated into liposomes using in vitro protein synthesis. A cryo-EM structure of the 16-helix transmembrane pore closely matches the design model. The ability to produce structurally and functionally well-defined transmembrane pores opens the door to the creation of designer pores for a wide variety of applications.

Introduction

De novo design of transmembrane proteins with large pores presents a stringent thermodynamic challenge, as there is a larger surface area to volume ratio and hence a lower density of stabilizing interactions. A homo-oligomeric architecture in which a cyclic arrangement of multiple identical copies of a single subunit surrounds the pore is attractive for design as the subunit need not be a large protein. The transformation of soluble oligomeric protein pores into their membrane counterparts is challenging because of the altered thermodynamics of folding in the membrane and possible undesired interactions between non-polar residues introduced to interact with lipids and those at intersubunit interfaces.

We set out to design transmembrane protein pores using a two-step approach. We reasoned that protein pores formed from two concentric rings of α-helices containing buried hydrogen bond networks for structural specificity could be stable in both soluble and, after resurfacing the lipid exposed residues with membrane-compatible hydrophobic residues, transmembrane forms. The increased interhelical interaction surfaces in such two-ring bundles could provide greater stability than single-ring structures, particularly for larger pore sizes, and could act together with buried hydrogen bond networks to yield greater structural robustness to conversion between soluble and transmembrane forms. We then sought to design cylindrical two-ring bundles consisting of a minimum of 12 closely packed helices that are assembled from 2-4 helix monomers, to avoid having to build such large structures from a single polypeptide.

### Designing an Ion Channel

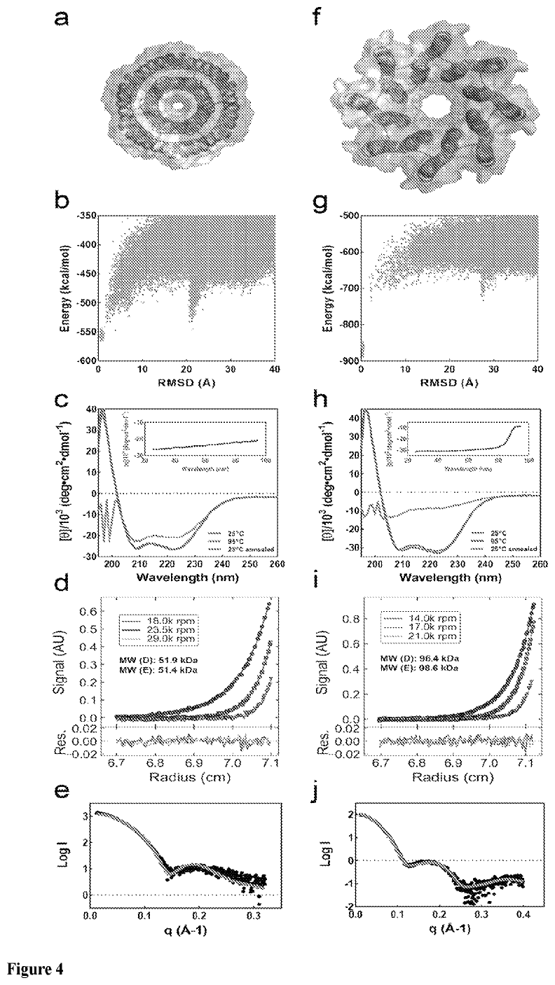

We explored the design of water-soluble pores containing helical bundles with a two-ring 6-fold symmetric topology formed from monomeric subunits composed of an inner and outer helix bridged with a short loop. Backbones were generated by sampling α-helical and superhelical parameters of the assemblies using a generalization of the Crick coiled-coil parameterization'. For each topology, the α-helical phase, superhelical radius, superhelical twist, and axial offset between helices were allowed to vary, while the superhelical phase was predetermined by the symmetry (360/6 =60° for the hexamer). After backbone generation and loop closure, we searched for hydrogen-bond networks across intermolecular interfaces using Rosetta™ HBNet and carried out combinatorial sequence optimization for the remaining residue positions keeping the polar networks found by HBNet fixed. The core residues and backbone parameters of the inner helices were set to those of a previously characterized “single-ring” helical bundle to reduce the design space. Rosetta™ “fold-and-dock” structure prediction calculations were used to investigate the extent to which the designed sequences encode the design target topologies. Designs (FIG. 4a) for which the lowest energy sampled structures were close to the target design structures (FIG. 4b) were selected for experimental characterization.

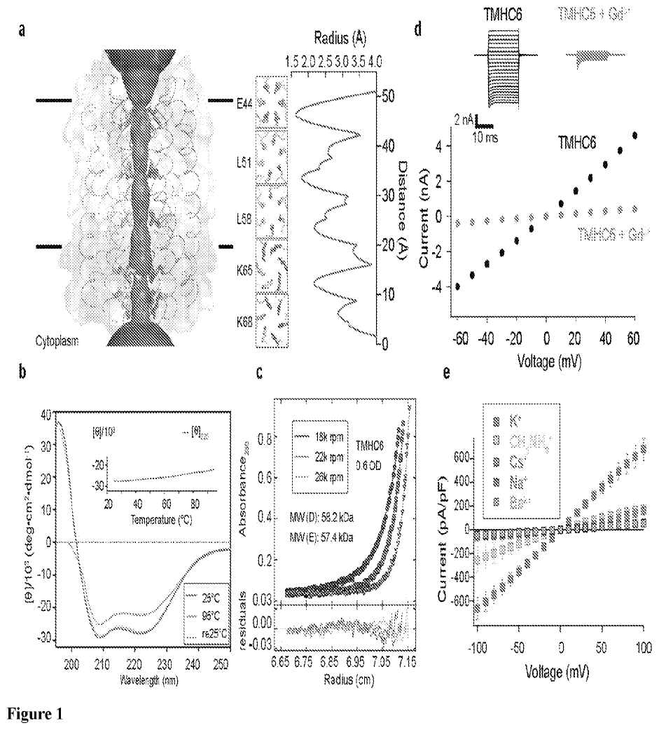

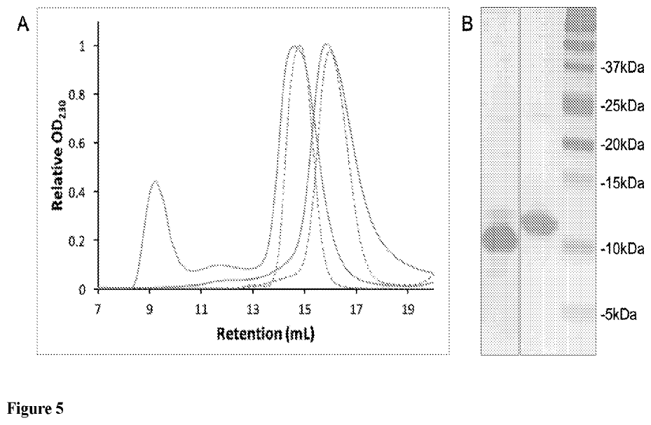

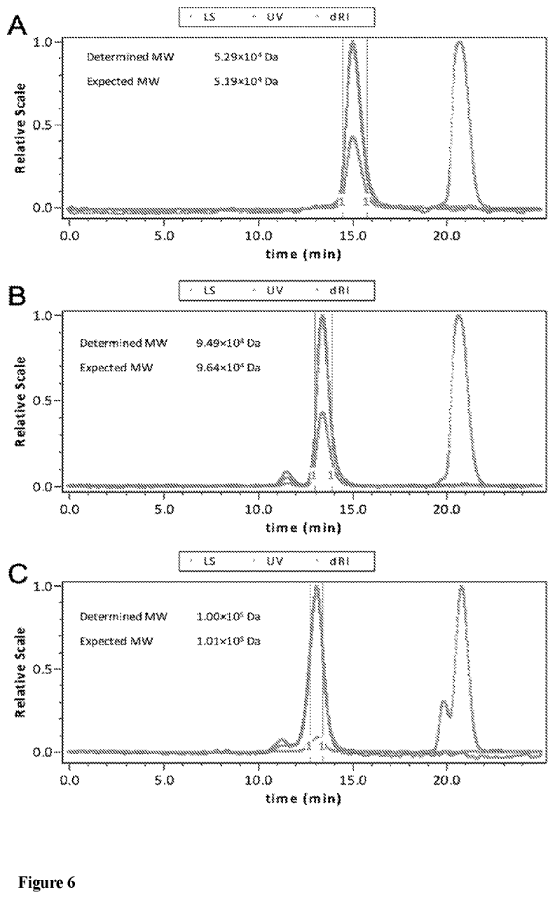

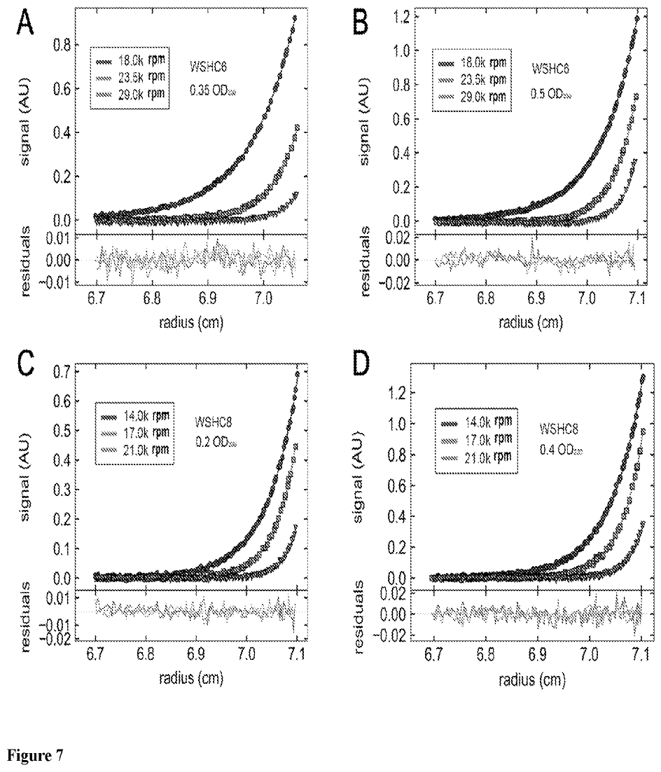

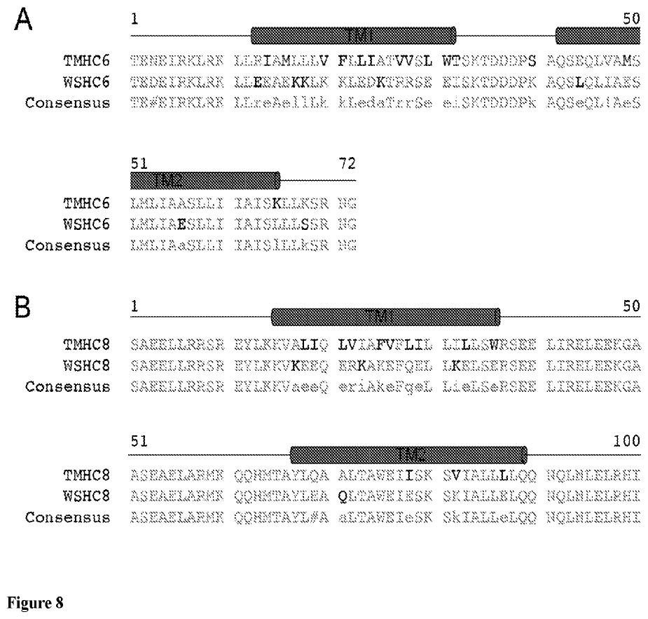

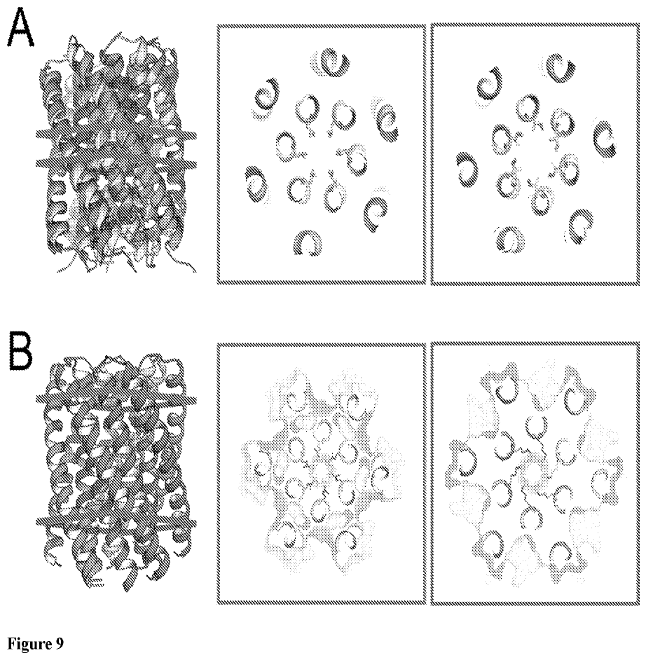

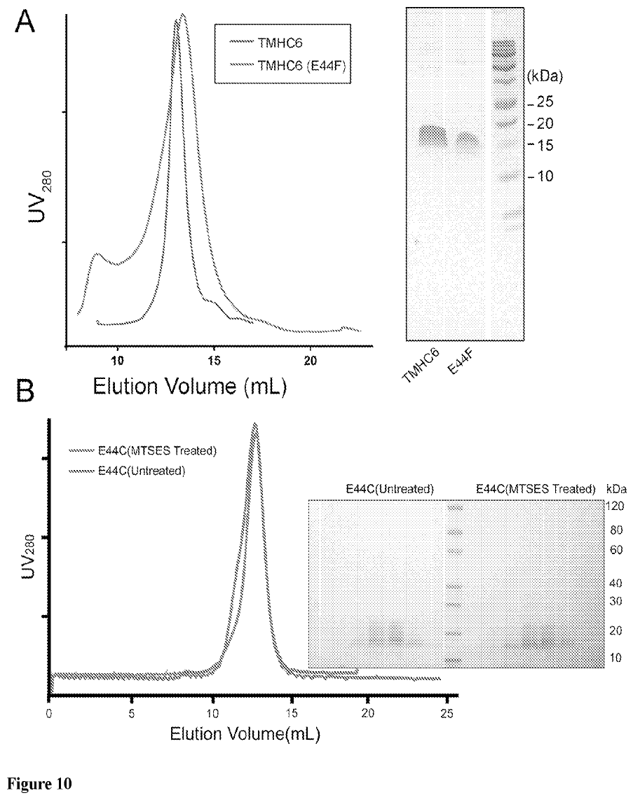

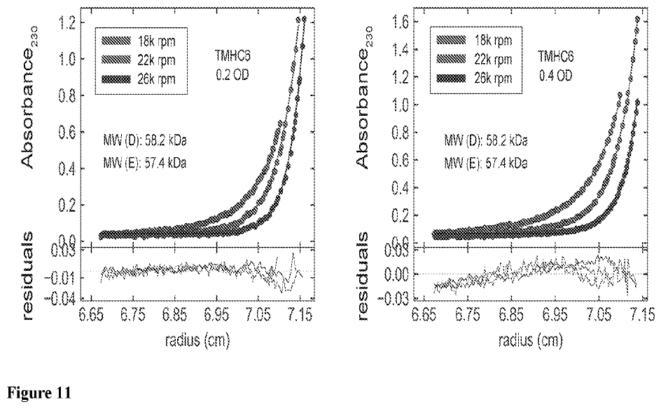

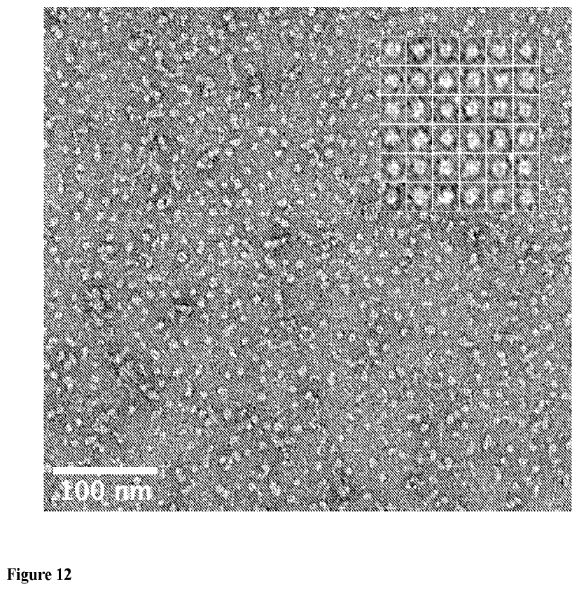

We obtained synthetic genes encoding the selected designs and expressed them in Escherichia coli (E. coli). All 3 hexameric designs selected for experimental validation were well expressed and soluble in E. coli and purified efficiently by nickel-affinity chromatography and size-exclusion chromatography (SEC, FIG. 5). One of the designs was found using multiangle light scattering (MALS, FIG. 6) and analytical ultracentrifugation (FIG. 4d, 7) to form a single homogeneous species with the target oligomeric state. The circular dichroism (CD) spectrum of this hexameric design (WSHC6, water-soluble hairpin C6) was highly helical consistent with the design model (FIG. 4c); WSHC6 was stable to thermal denaturation up to 95° C. The small-angle X-ray scattering (SAXS) profile of WSHC6 was close to that computed from the design model, suggesting that WSHC6 folds into the desired shape in solution (FIG. 4e). We determined the crystal structure of WSHC6 and found it to be close to the computational design model (data not shown) with a C-a RMSD of 0.89 Å. A chain of discrete water molecules occupies the WSHC6 channel; the narrowest constriction is at Leu51 with a diameter of approximately 4 Å as calculated by HOLE^{TM20}. We next sought to convert the stable and structurally well-defined WSHC6 pore into a transmembrane hexameric pore (TMHC6, transmembrane hairpin C6). We redesigned the lipid exposed residues (see Supplementary Information) and incorporated one ring of six glutamate residues (E-ring) and two rings of lysine residues (K-rings) at the openings of the central channel on the extracellular and intracellular side, respectively, to create a polar niche within the pore (FIG. 1a, 8, 9). The narrowest regions of the TMHC6 pore in the design model are the E-ring (3.3 Å), the K-ring (4.3 Å), and two intervening rings of hydrophobic leucine residues (L-rings, 3.8 Å) (FIG. 1a). A synthetic gene was obtained for TMHC6 and the protein was expressed in E. coli, extracted from the membrane fraction with detergent, and purified by affinity chromatography and SEC (FIG. 10). TMHC6 eluted as a single peak upon SEC, and CD measurements showed that the design is α-helical and highly thermal stable with a CD spectrum at 95° C. similar to that at 25° C. The protein sedimented as a hexamer in detergent solution in analytical ultracentrifugation (AUC) experiments, consistent with the design model (FIG. 1c, 11). Electron microscopy of negatively stained samples showed populations of particles with shape and size consistent with the soluble protein crystal structure and the design model (FIG. 12).

To investigate ion conductance by TMHC6, we performed whole-cell patch clamp experiments on Trichoplusia ni insect cells (Hi5) expressing the designed transmembrane proteins. Using extracellular (bath) and intracellular (electrode) solutions containing 100 mM NaCl and 100 mM NaF, respectively, the TMHC6 construct exhibited a symmetric current/voltage relationship for inward and outward Na current as a function of membrane potential (FIG. 1d). Gadolinium ion (Gd³+), a potent blocker of cation channels, blocked TMHC6 from the extracellular side, reducing the ion conductance to nearly the control value for untransfected cells (FIG. 1d). To test the ion selectivity, Hi5 cells expressing TMHC6 were bathed in a solution containing 100 mM of the chloride salt of the monovalent cations K+, Na+, Cs+, and CH3NH3+. TMHC6 showed a significantly higher conductance for K⁺, with a current density of 600 pA/pF at +100 mV. The selectivity order was K+(600 pA/pF) >>Cs⁺═CH₃NH₃⁺ (170 pA/pF)>Na⁺ (60pA/pF)≈Ba²⁺ (54 pA/pF) (FIG. 1e).

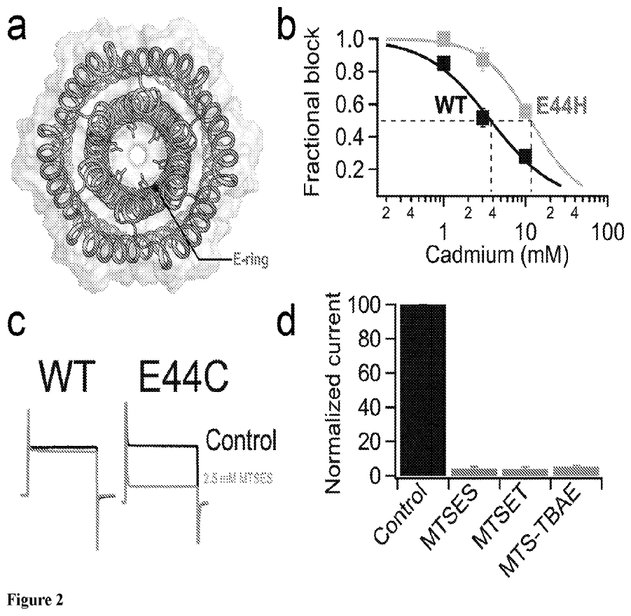

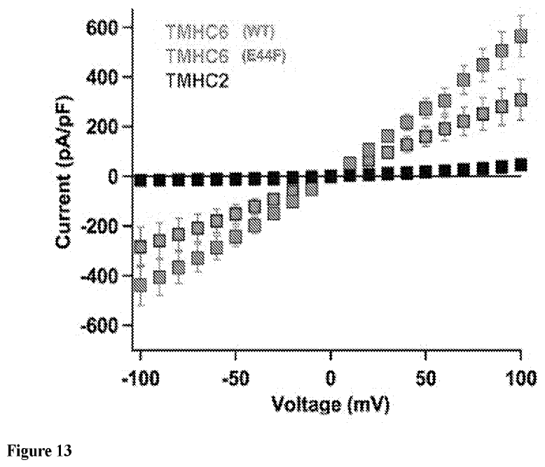

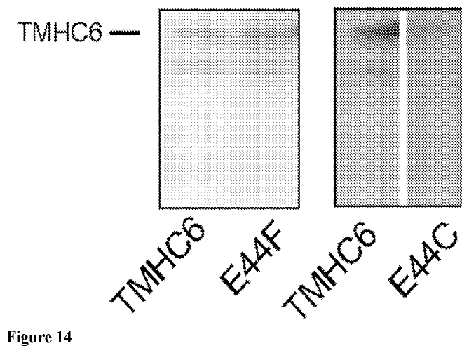

In the design model, the extracellular ring of six Glu44 residues is the site of cation entry; we tested this hypothesis by site-directed mutagenesis and chemical modification. Mutation of Glu44 to Phe (E44F) removed the negative charge at the extracellular entry to the pore and reduced the conductance to 308±81 pA/pF, 51.3% of the control (FIG. 13). This reduction likely is a direct effect on ion traversal through the pore: the E44F mutant is expressed at a similar level as the TMHC6 parent (FIG. 14), and the purified protein has a similar elution volume as TMHC6 (FIG. 10a). To further test the importance of this site, we constructed the mutant E44H, which deletes the negative charge in this position and adds a partial positive charge. The divalent cation Cd²⁺ blocks the conductance of TMHC6, and 3-fold higher Cd²⁺ concentrations were required to block the E44H mutant, likely because of the reduced electrostatic attraction and/or disrupted metal coordination (FIG. 2b). To enable a chemical biology approach, we constructed the mutant E44C, which removes the negative charge at the extracellular entry to the pore and reduced the conductance to 360±36 pA/pF, 60% of the control. The chemical reactivity of the substituted Cys residue allowed us to test sulfhydryl reagents as pore blockers. We found that perfusion of negatively charged MTSES, positively charged MTSET, and hydrophobic MTS-TBAE all completely blocked the ion conductance of the TMHC6 E44C mutant within a few minutes under voltage clamp control (FIG. 2c and d). The lack of dependence on charge or hydrophilicity suggests that these compounds function by directly sterically blocking the pore. The blockage by these reagents is entirely dependent on the introduced cysteine residue; they had no effect on the original TMHC6 design lacking the cysteine (FIG. 2c and d). To determine whether covalent modification had any global effect on the folding or assembly of the pore, we expressed and purified the TMHC6 E44C mutant from E. coli and incubated it with MTSES. Covalent modification was observed by mass spectrometry (data not shown), but there was no change in pore assembly or solution behavior more generally (FIG. 10b), further suggesting that chemical modification blocks ion conductance by direct steric occlusion.

Taken together, the high ion selectivity, specific inhibition by multivalent cations, and complete block by MTS reagents acting at the extracellular entry to the pore strongly suggest that ion passage is through the designed central transmembrane pore. Leak conductances would not be expected to have these properties. Single channel recordings on TMHC6 did not yield clear signals. The single channel conductance may be too low, perhaps because of the central non-polar lined portion of the pore.

Why is TMHC6 selective for K⁺? The TMHC6 pore diameter of 3.3 Å is well tuned for the conductance of dehydrated K⁺. In contrast, voltage-gated sodium and calcium channels, which conduct hydrated Na and Ca' have pores that are 4.6 Å in diameter, well designed for hydrated sodium and calcium ions but not for K⁺. The ability to design transmembrane pores de novo lays the foundation for broad exploration of the pore diameters and chemical interactions that are required for selective conductance of a wide range of ions. This understanding enables the design of channels which directly modulate cell function. Towards that end, we expressed TMHC6 in a yeast strain that requires potassium for growth, and we found that TMHC6 considerably boosted the growth rate in a K⁺ dependent fashion (data not shown).

### Building a Larger Transmembrane Nanopore

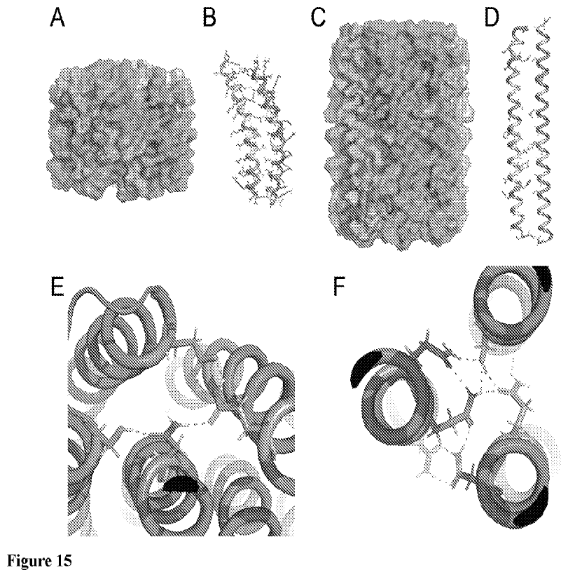

To explore the generation of larger transmembrane pores capable of transporting organic molecules larger than single ions, we designed water-soluble helical bundles with 8-fold symmetry using an approach similar to that described above for WSHC6 except that a starting “single-ring” template was not employed; instead, the structure and sequence of the inner ring of helices was sampled de novo in parallel with those of the outer ring. We obtained synthetic genes encoding selected octameric pore designs and expressed them in E. coli. Four of five of the octameric designs were well expressed and soluble in E. coli, and they could be purified by nickel-affinity chromatography and SEC. However, none of the designs populated only the target octameric state. There are only small differences in interface geometry between C7, C8 and C9 assemblies (the angles between subunits are 51°, 45° and 40°, respectively), and mixtures were observed for most of the designs. We reasoned that achieving a well-defined C8 state would require more precise interface definition; therefore, we carried out a second round of C8 designs with larger inter-subunit interaction surface areas and more hydrogen bond networks across the interface (FIG. 15). Ten of 15 of the second-round designs were expressed and soluble in E. coli, and two were found to be mono-disperse and octameric by MALS (FIG. 6). One of the two was further confirmed by AUC (FIG. 4i, 7). The CD spectrum of the AUC-verified octameric design (WSHC8, water-soluble hairpin C8) was highly helical consistent with the design model (FIG. 4h). The design is also quite stable with a melting temperature of 85° C. (FIG. 4h insets). There was again excellent agreement of the experimental and calculated SAXS profiles indicating that WSHC8 folds into the desired shape in solution (FIG. 4j).

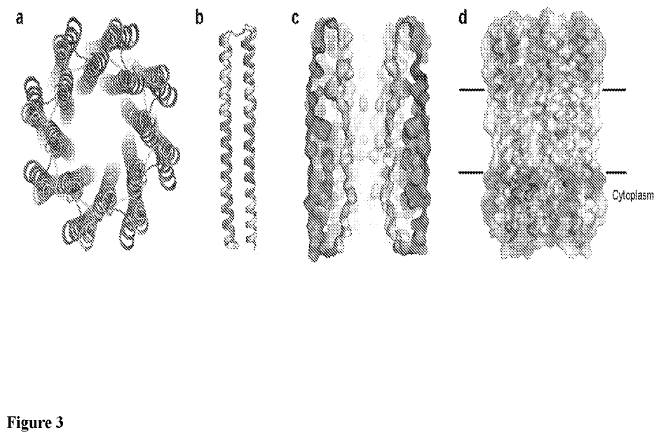

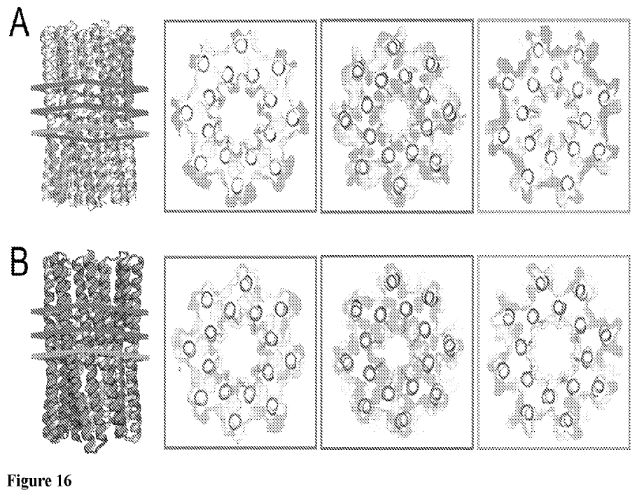

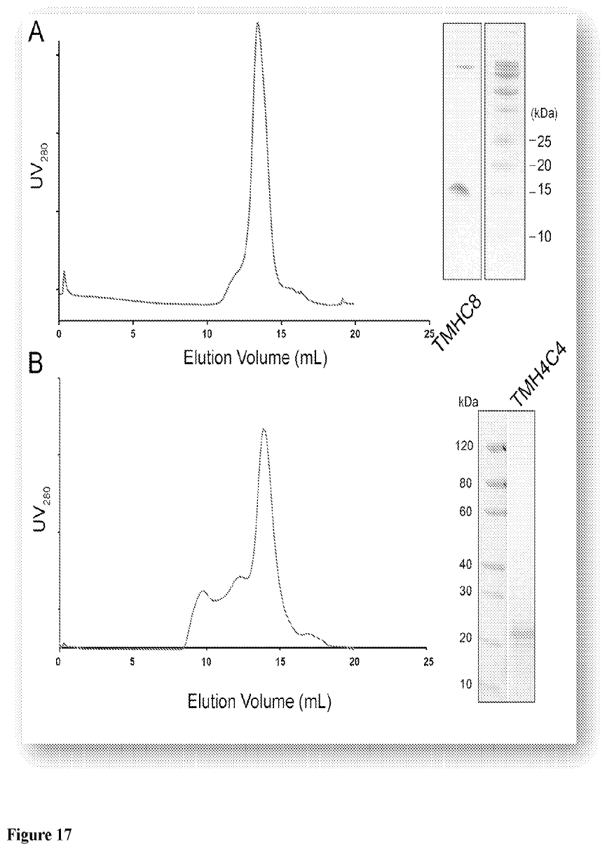

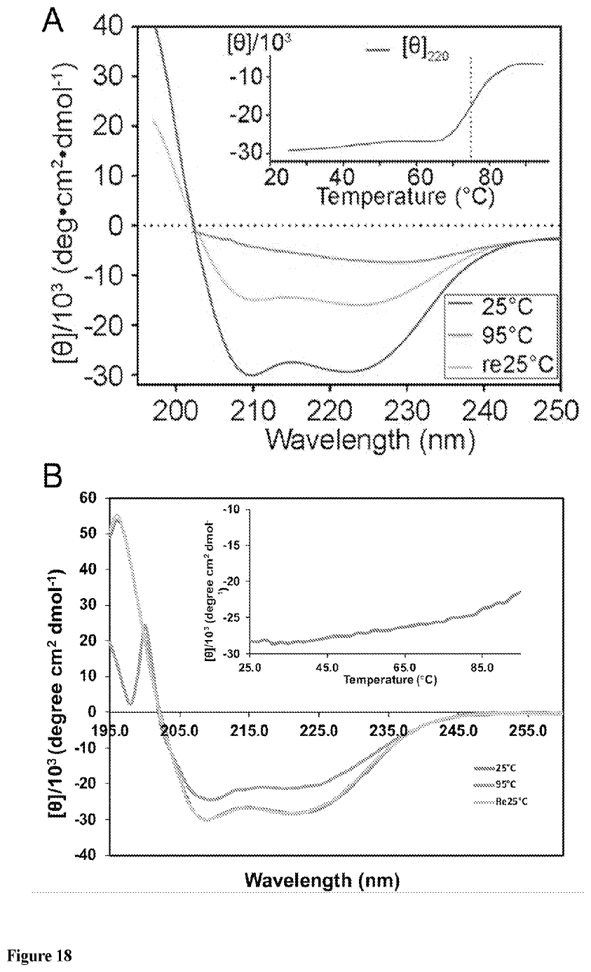

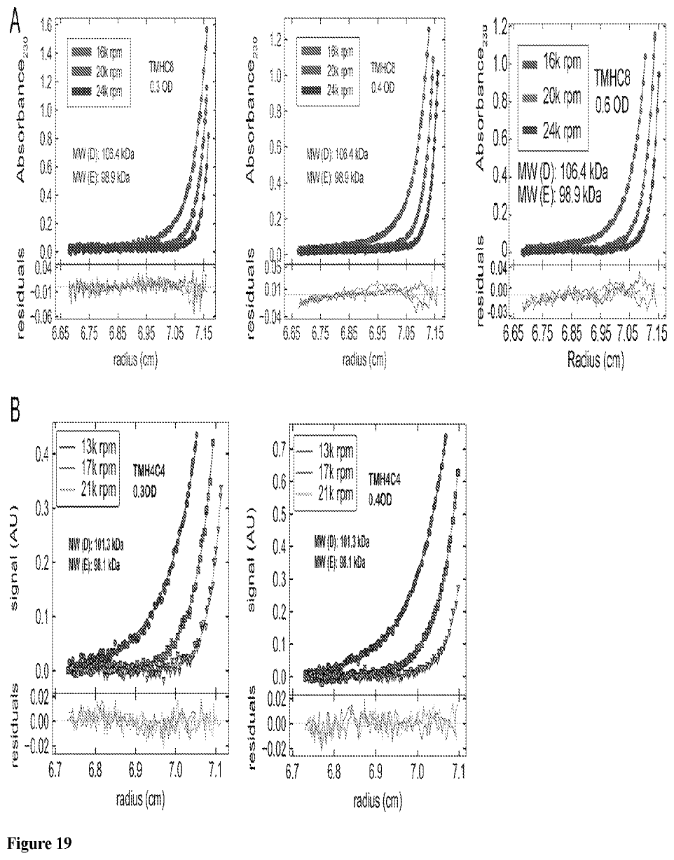

We determined the crystal structure for WSHC8 (FIG. 3a and b) and found that the C-α RMSD values between the crystal structure and the design model for the monomeric subunit and the full octameric pore were 0.97 Å and 2.51 Å, respectively. The larger deviation compared to WSHC6 is caused by the slight tilting of the hairpin monomers along the superhelical axis of the complex (FIG. 16a). As in the design models, the structure contains a long and continuous central channel with an inner diameter of approximately 10 Å as calculated by HOLE™ (FIG. 3c, 16). We converted WSHC8 into an octameric membrane-spanning pore (TMHC8) by redesigning the membrane exposed and pore facing residues of the crystal structure (see Supplementary Information). The design model has a central pore with a diameter of 10 Å and a transmembrane span of 31 Å. A gene encoding TMHC8 was synthesized, the protein expressed in E. coli, and the membrane fraction purified and solubilized in detergent. Following affinity chromatography, the protein eluted as a monodisperse peak on SEC (FIG. 17a). CD measurements showed that TMHC8 had the expected α-helical secondary structure with a melting temperature of 75° C. (FIG. 18a). AUC experiments showed that TMHC8 formed complexes with a molecular weight (MW) of 98.8 kDa, which lies in between the MWs of a 14-helix heptamer and a 16-helix octamer (FIG. 19a). To resolve this ambiguity, we linked two monomers of TMHC8 together using a short loop to create a four-helix subunit with two inner ring helices and two outer ring helices (FIG. 3d). This redesign, TMH4C4, was purified to homogeneity using nickel-affinity chromatography and SEC (FIG. 17b). CD spectroscopy showed that TMH4C4 was fully α-helical and thermally stable up to 95° C. (FIG. 18b). AUC experiments showed that TMH4C4 sedimented as a tetramer in detergent solution consistent with the 16-helix design model (FIG. 19b).

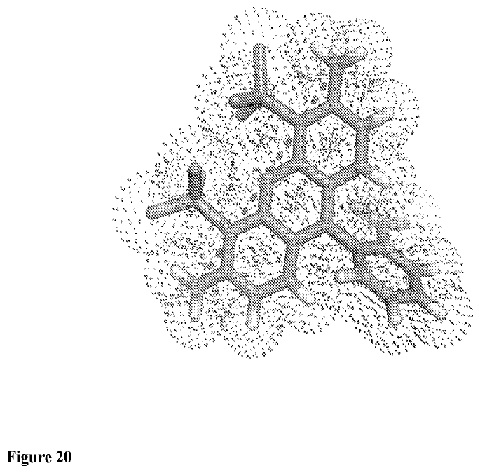

Expression of the designed larger transmembrane nanopore in insect cells resulted in cell death, probably because of induced cell permeability, and hence we were unable to assess the channel activity in these cells. Instead, we used a liposome-based assay coupled to in vitro protein synthesis²⁸⁻³⁰. TMHC6 and TMH4C4 were produced inside liposomes containing streptavidin, and biotinylated Alexa™ Fluor 488 (MW ˜1 kDa) was added outside (data not shown). In vitro synthesized TMHC6 and TMH4C4 had similar elution volumes as the bacterially expressed and purified proteins when analyzed by SEC (data not shown). Consistent with the much larger pore radius of TMH4C4 compared to TMHC6, we observed considerably more accumulation of dye within proteoliposomes containing TMH4C4 than in proteoliposomes containing TMHC6, or empty liposomes (we controlled for differences in protein expression and membrane incorporation of the two proteins by adjusting the template DNA concentration such that the amount of protein present in the membrane was the same). The narrowest dimension of the head group of the fluorophore is approximately 12 Å (FIG. 20), while the nominal diameter of the constriction region of the TMH4C4 pore is estimated to be 10 Å by HOLE™, thermal fluctuations of the side chains and backbone likely allow for permeation by the fluorophore. In contrast, the 3.3 521 constriction of TMHC6 is far too narrow to allow fluorophore passage. Increasing the size of the fluorescent conjugate to 4.6 kDa by inserting poly adenine oligo DNA (A11) between Alexa™ Fluor 488 and biotin blocked passage through both the TMHC6 or TMH4C4 pores (data not shown), consistent with the estimated hydrodynamic diameter of this compound of 30 Å³¹.

Seeking to obtain an overall structure of the 16-helix transmembrane channel, we used cryogenic electron microscopy (cryo-EM) to determine the three-dimensional (3D) structure of TMH4C4. The protein was concentrated to ˜6 mg/ml, applied to EM grids, and cryo-EM images collected and processed following standard protocols (data not shown). To avoid potential bias, Cl symmetry was applied for automatic image processing and classification, which yielded a dominant 16-helix form containing ˜40% of all the 3D classified particles, with the most continuous and intact map among all classes. Further classification and refinement focused on this set of particles resulted in a 5.9 Å resolution map from 64,739 (out of a total of 1,559,110 for 3D classification) selected particles (data not shown). The cryo-EM structure clearly shows the formation of a 16-helix assembly with a large central pore, consistent with the design model built from the crystal structure of the soluble form. Density encircling the membrane-spanning region likely originates from surrounding detergent molecules. A structure model of TMH4C4 built based on the EM map had some deviation among the four protomers so the tetramer is not perfectly symmetric, but the central pore-containing 16-helix structure corresponding to the original TMH4C4 design is clearly resolved, and aligns well to the design model.

### Conclusion

Our advances in designing structurally well-defined transmembrane pores, both inform our understanding of general principles of protein biophysics, and open the door for a wide range of applications. From the membrane protein folding perspective, our success in designing transmembrane homo-oligomeric structures with hydrogen bond network containing subunit interfaces suggests that the combination of the substantial buried surface area and polarity of the interaction surfaces makes them robust to changes in the surrounding environment and reduces confounding by interactions with the purely hydrophobic lipid facing residues during folding. This approach allows the construction of quite substantial pores with environments very different from that of the surrounding lipids: the TMHC6 pore clearly shows selective transmembrane ion conductance, and the 10 Å diameter TMH4C4 pore clearly evident in the cryo-EM structure is lined with polar residues and provides passage to solutes as large as biotinylated Alexa™ Fluor 488.

Custom design now provides a route to understanding the underlying chemistry and physics of solute permeation and selectivity by modulating pore structures and selectivity filters in ways that are not possible with native channels. Our strategy of first designing soluble pore-containing structures, and then converting the stable designs into transmembrane proteins following crystal structure determination, leverages the considerably more straightforward structural characterization of soluble proteins as a key step toward building complex transmembrane proteins with a high success rate (see the Supplementary Information). Additional selectivity filters and gating mechanisms can be incorporated both to test basic concepts and to enable a wide range of applications. For example, in nanotechnology, custom designed pores could provide new routes to generating membranes with selective permeabilities, sensing molecules in the environment, and controlling cellular behavior.

### REFERENCES

1. Gilbert, R. J. C., Bayley, H. & Anderluh, G. Membrane pores: from structure and assembly, to medicine and technology. Philos. Trans. R. Soc. Lond. B Biol. Sci. 372, (2017).
2. Eisenstein, M. An ace in the hole for DNA sequencing. Nature 550, 285-288 (2017).
3. Kasianowicz, J. J., Brandin, E., Branton, D. & Deamer, D. W. Characterization of individual polynucleotide molecules using a membrane channel. Proc. Natl. Acad. Sci. US.A. 93, 13770-13773 (1996).
4. Clarke, J. et al. Continuous base identification for single-molecule nanopore DNA sequencing. Nat. Nanotechnol. 4, 265-270 (2009).
5. Lear, J. D., Wasserman, Z. R. & DeGrado, W. F. Synthetic amphiphilic peptide models for protein ion channels. Science 240, 1177-1181 (1988).
6. Akerfeldt, K. S., Lear, J. D., Wasserman, Z. R., Chung, L. A. & DeGrado, W. F.

Synthetic peptides as models for ion channel proteins. Acc. Chem. Res. 26, 191-197 (1993).
7. Joh, N. H. et al. De novo design of a transmembrane Zn²⁺-transporting four-helix bundle. Science 346, 1520-1524 (2014).
8. Lu, P. L. et al. Accurate computational design of multipass transmembrane proteins. Science 359, 1042-1046 (2018).
9. Mahendran, K. R. et al. A monodisperse transmembrane α-helical peptide barrel. Nat. Chem. 9, 411-419 (2017).
10. Mravic, M. et al. Packing of apolar side chains enables accurate design of highly stable membrane proteins. Science 363, 1418-1423 (2019).
11. Joh, N. H., Grigoryan, G., Wu, Y. & DeGrado, W. F. Design of self-assembling transmembrane helical bundles to elucidate principles required for membrane protein folding and ion transport. Philos. Trans. R. Soc. Lond. B Biol. Sci. 372, (2017).
12. Niitsu, A., Heal, J. W., Fauland, K., Thomson, A. R. & Woolfson, D. N. Membrane-spanning α-helical barrels as tractable protein-design targets. Philos. Trans. R. Soc. Lond. B Biol. Sci. 372, 20160213 (2017).
13. Thomson, A. R. et al. Computational design of water-soluble α-helical barrels. Science 346, 485-488 (2014).
14. Rhys, G. G. et al. Maintaining and breaking symmetry in homomeric coiled-coil assemblies. Nat. Commun. 9, 4132 (2018).
15. Crick, F. H. C. The Fourier transform of a coiled-coil. Acta Crystallogr. 6, 685-689 (1953).
16. Grigoryan, G. & Degrado, W. F. Probing designability via a generalized model of helical bundle geometry. J. Mol. Biol. 405, 1079-1100 (2011).
17. Huang, P. S. et al. High thermodynamic stability of parametrically designed helical bundles. Science 346, 481-485 (2014).
18. Boyken, S. E. et al. De novo design of protein homo-oligomers with modular hydrogen-bond network-mediated specificity. Science 352, 680-687 (2016).
19. Das, R. et al. Simultaneous prediction of protein folding and docking at high resolution. Proc. Natl. Acad. Sci. U.S.A. 106, 18978-18983 (2009).
20. Smart, O. S., Neduvelil, J. G., Wang, X., Wallace, B. A. & Sansom, M. S. P. HOLE: A program for the analysis of the pore dimensions of ion channel structural models. J. Mol. Graph. 14, 354-360 (1996).
21. Hou, X., Pedi, L., Diver, M. M. & Long, S. B. Crystal structure of the calcium release-activated calcium channel Orai. Science 338, 1308-1313 (2012).
22. Hou, X., Burstein, S. R. & Long, S. B. Structures reveal opening of the store-operated calcium channel Orai. Elife 7, (2018).
23. Dynes, J. L., Amcheslaysky, A. & Cahalan, M. D. Genetically targeted single-channel optical recording reveals multiple Orail gating states and oscillations in calcium influx. Proc. Natl. Acad. Sci. U S. A. 113, 440-445 (2016).
24. Jiang, Y. et al. X-ray structure of a voltage-dependent K+ channel. Nature 423, 33-41 (2003).
25. Payandeh, J., Scheuer, T., Zheng, N. & Catterall, W. A. The crystal structure of a voltage-gated sodium channel. Nature 475, 353-358 (2011).
26. Tang, L. et al. Structural basis for Ca2+ selectivity of a voltage-gated calcium channel. Nature 505, 56-61 (2014).
27. Pan, X. et al. Structure of the human voltage-gated sodium channel Nav1.4 in complex with β1. Science 362, (2018).
28. Fujii, S. et al. Liposome display for in vitro selection and evolution of membrane proteins. Nat. Protoc. 9, 1578-1591 (2014).
29. Fujii, S., Matsuura, T., Sunami, T., Kazuta, Y. & Yomo, T. In vitro evolution of α-hemolysin using a liposome display. Proc. Natl. Acad. Sci. U S. A. 110, 16796-16801 (2013).
30. Dwidar, M. et al. Programmable Artificial Cells Using Histamine-Responsive Synthetic Riboswitch. J. Am. Chem. Soc. 141, 11103-11114 (2019).
31. Sim, A. Y. L., Lipfert, J., Herschlag, D. & Doniach, S. Salt dependence of the radius of gyration and flexibility of single-stranded DNA in solution probed by small-angle x-ray scattering. Phys. Rev. E Stat. Nonlin. Soft Matter Phys. 86, 021901 (2012).
32. Huang, P.-S., Boyken, S. E. & Baker, D. The coming of age of de novo protein design. Nature 537, 320-327 (2016).
33. Song, L. et al. Structure of staphylococcal alpha-hemolysin, a heptameric transmembrane pore. Science 274, 1859-1866 (1996).
34. Pettersen, E. F. et al. UCSF Chimera?A visualization system for exploratory research and analysis. Journal of Computational Chemistry vol. 25 1605-1612 (2004).
35. Chen, Z. et al. Programmable design of orthogonal protein heterodimers. Nature 565, 106-111 (2019).
36. Krogh, A., Larsson, B., von Heijne, G. & Sonnhammer, E. L. Predicting transmembrane protein topology with a hidden Markov model: application to complete genomes. J. Mol. Biol. 305, 567-580 (2001).
37. Studier, F. W. Protein production by auto-induction in high density shaking cultures. Protein Expr. Purif 41, 207-234 (2005).
38. Cole, J. L., Lary, J. W., P Moody, T. & Laue, T. M. Analytical ultracentrifugation: sedimentation velocity and sedimentation equilibrium. Methods Cell Biol. 84, 143-179 (2008).
39. Dyer, K. N. et al. High-throughput SAXS for the characterization of biomolecules in solution: a practical approach. Methods Mol. Biol. 1091, 245-258 (2014).
40. Franke, D. et al. ATSAS 2.8: a comprehensive data analysis suite for small-angle scattering from macromolecular solutions. J. Appl. Crystallogr. 50, 1212-1225 (2017).
41. Svergun, D., Barberato, C. & Koch, M. H. J. CRYSOL--a program to evaluate X-ray solution scattering of biological macromolecules from atomic coordinates. J. Appl. Crystallogr. 28, 768-773 (1995).
42. Adams, P. D. et al. PHENIX: a comprehensive Python-based system for macromolecular structure solution. Acta Crystallogr. D Biol. Crystallogr. 66, 213-221 (2010).
43. McCoy, A. J. et al. Phaser crystallographic software. J Appl. Crystallogr. 40, 658-674 (2007).
44. Terwilliger, T. C. et al. phenix.mr rosetta: molecular replacement and model rebuilding with Phenix and Rosetta. J. Struct. Funct. Genomics 13, 81-90 (2012).
45. Emsley, P., Lohkamp, B., Scott, W. G. & Cowtan, K. Features and development of Coot. Acta Crystallogr. D Biol. Crystallogr. 66, 486-501 (2010).
46. Minor, D. L., Masseling, S. J., Jan, Y. N. & Jan, L. Y. Transmembrane Structure of an Inwardly Rectifying Potassium Channel. Cell vol. 96 879-891 (1999).
47. Tang, W. et al. Functional expression of a vertebrate inwardly rectifying K+ channel in yeast. Mol. Biol. Cell 6, 1231-1240 (1995).
48. Pautot, S., Frisken, B. J. & Weitz, D. A. Production of Unilamellar Vesicles Using an Inverted Emulsion. Langmuir 19, 2870-2879 (2003).
49. Nishimura, K. et al. Cell-free protein synthesis inside giant unilamellar vesicles analyzed by flow cytometry. Langmuir 28, 8426-8432 (2012).
50. Nishimura, K. et al. Population analysis of structural properties of giant liposomes by flow cytometry. Langmuir 25, 10439-10443 (2009).
51. Soga, H. et al. In vitro membrane protein synthesis inside cell-sized vesicles reveals the dependence of membrane protein integration on vesicle volume. ACS Synth. Biol. 3, 372-379 (2014).
52. Matsuura, T., Hosoda, K., Ichihashi, N., Kazuta, Y. & Yomo, T. Kinetic analysis of β-galactosidase and β-glucuronidase tetramerization coupled with protein translation. J. Biol. Chem. 286, 22028-22034 (2011).
53. Lei, J. & Frank, J. Automated acquisition of cryo-electron micrographs for single particle reconstruction on an FEI Tecnai electron microscope. J. Struct. Biol. 150, 69-80 (2005).
54. Zheng, S. Q. et al. MotionCor2: anisotropic correction of beam-induced motion for improved cryo-electron microscopy. Nat. Methods 14, 331-332 (2017).
55. Grant, T. & Grigorieff, N. Measuring the optimal exposure for single particle cryo-EM using a 2.6 Å reconstruction of rotavirus VP6. Elife 4, e06980 (2015).
56. Zhang, K. Gctf: Real-time CTF determination and correction. J. Struct. Biol. 193, 1-12 (2016).
57. Zivanov, J. et al. New tools for automated high-resolution cryo-EM structure determination in RELION-3. Elife 7, (2018).
58. Rosenthal, P. B. & Henderson, R. Optimal determination of particle orientation, absolute hand, and contrast loss in single-particle electron cryomicroscopy. J. Mol. Biol. 333, 721-745 (2003).
59. Goddard, T. D., Huang, C. C. & Ferrin, T. E. Visualizing density maps with UCSF Chimera. J. Struct. Biol. 157, 281-287 (2007).
60. Conway, P., Tyka, M. D., DiMaio, F., Konerding, D. E. & Baker, D. Relaxation of backbone bond geometry improves protein energy landscape modeling. Protein Sci. 23, 47-55 (2014).
61. DiMaio, F., Tyka, M. D., Baker, M. L., Chiu, W. & Baker, D. Refinement of protein structures into low-resolution density maps using rosetta. J. Mol. Biol. 392, 181-190 (2009).
62. Kazuta, Y., Matsuura, T., Ichihashi, N. & Yomo, T. Synthesis of milligram quantities of proteins using a reconstituted in vitro protein synthesis system. J. Biosci. Bioeng. 118, 554-557 (2014).

### Supplementary Materials

### 1. Computational Modeling

### 1.1 Design of Water-Soluble Proteins

### Systematic Sampling of Parametric Helical Backbones

Helical backbones used for design calculations were generated by sampling the generalized Crick coiled-coil parameters ^{15,16} as described previously^{17,18}. Only supercoil and, alpha-helical parameters of the inner and outer helices of the monomers were sampled. These parameters include supercoil twist (ω₀), supercoil radius (R₀), alpha-helical phase (φ₁), and the offset along the z-axis (Z_{offset}). The supercoil phases (φ₀) were fixed at 0° for the inner helices and −30° and −22.5° for the outer helices of hexamers and octamers, respectively. The supercoil pitch of the outer helices was constrained to match that of the inner helices to maintain contacts to the inner helices throughout the length of the helical bundles¹⁸. The Alpha-helical twists (ω₁) were fixed at ideal values of 102.85°/aa for left-handed supercoils (e.g. WSHC6) and 100°/aa for straight helical bundles (e.g. WSHC8). Cyclic symmetries were applied during design calculations. The parameters for the inner helices of WSHC6 were preset according to the fitting result of a previously reported six-helix bundle¹³ using the Coiled-coil Crick Parameterization web server. Supercoil and alpha-helical parameters used to generate the backbones of WSHC6 and WSHC8 (2nd & 3rd columns) and the fitted parameters for the final design model and the crystal structure of WSHC8 (4th & 5th columns) are shown in Table 1:

TABLE 1

| | WSHC6 bb | | WSHC8 bb | | WSHC8 | | WSHC8 | |
|---|---|---|---|---|---|---|---|---|
| | generation | | generation | | design model | | crystal structure | |
| number of residues of the inner helix | 34 | | 49 | | 49 | | 49 | |
| number of residues of the outer helix | 34 | | 49 | | 49 | | 49 | |
| supercoil twist ω₀ of the inner helix | −3.18°/aa | | 0°/aa | | −0.39°/aa | | −0.96°/aa | |
| supercoil twist ω₀ of the outer helix | −2.75°/aa | | 0°/aa | | −0.42°/aa | | −0.88°/aa | |
| alpha-helical twist ω₁ of the inner helix | 102.85°/aa | | 100.00°/aa | | 99.70°/aa | | 99.64°/aa | |
| alpha-helical twist ω₁ of the outer helix | 102.85°/aa | | 100.00°/aa | | 100.16°/aa | | 100.18°/aa | |
| supercoil radius R₀ of the inner helix | 9.5 | Å | 14.2 | Å | 14.4 | Å | 14.6 | Å |
| supercoil radius R₀ of the outer helix | 18.5 | Å | 23.2 | Å | 22.8 | Å | 23.3 | Å |
| supercoil phase φ₀ of the inner helix | 0° | | 0° | | 0° | | 0° | |
| supercoil phase φ₀ of the outer helix | −30° | | −22.5° | | −22.5° | | −22.5° | |
| alpha-helical phase φ₁ of the inner helix | 45.8° | | −98° | | −93° | | −94° | |
| alpha-helical phase φ₁ of the outer helix | 152.8° | | 74° | | 71° | | 65° | |
| z-axis offset Z_{offset} of the inner helix | 0 | Å | 0 | Å | 0 | Å | 0 | Å |
| z-axis offset Z_{offset} of the outer helix | 2.2 | Å | −3.2 | Å | −2.0 | Å | −1.9 | Å |

We learned from the failed first round of design of helical bundles with large pores that enforcing supercoil twist and knobs-into-holes packing of conventional left-handed coiled coils will cause a big curvature of the generated helices, as shown in FIG. 15A-B. In this case, precisely designed inter-subunit interactions may be required to compensate for the strain associated with curving the helices, which, therefore, makes designing large pore-containing helical proteins more challenging. In the second round of design, we fixed the supercoil twist at 0°/aa and saw a better success rate. Although the helices in the crystal structure of WSHC8 are more tilted compared to the design model, the supercoil twist is still much smaller than those of the conventional coiled coils and does not cause helix curving.

### Connecting Parametric Helical Backbones:

The parametrically generated inner and outer helical backbones were connected before design calculations with short 2-5-residue loops using methods described previously^{18,35}.

### Rosetta™ Design Calculations:

Cyclic symmetries were applied at the beginning of the calculations. The backbone geometry was then regularized by minimization in Cartesian space. HBNet¹⁸ was used to search for hydrogen-bond networks that span the helical interfaces. With atom pair constraints applied to the newly identified hydrogen-bond networks, Monte Carlo rotamer packing and minimization were performed to design the remaining positions aiming to lower the total energy and optimize the hydrophobic packing around the hydrogen-bond networks. A final minimization and side-chain repacking step was carried out without atom pair restraints on hydrogen-bonding residues to evaluate how well the networks remained intact in the absence of the constraints. Low energy designs with intact hydrogen-bond networks were tested using Rosetta™ “fold-and-dock” structure prediction calculations¹⁹. Sequences for which the predicted lowest-energy structures converge on the design models without additional energy minima were selected for experimental characterizations (Table 2).

TABLE 2
Sequences of designed proteins

| | |
|---|---|
| WSHC6 (SEQ ID NO: 5) | TEDEIRKLRKLLEEAEKKLY |
| | KLEDKTRRSEEISKTDDDPK |
| | AQSLQLIAESLMLIAESLLI |
| | IAISLLLSSRNG |
| WSHC8 (SEQ ID NO: 2) | SAEELLRRSREYLKKVKEEQ |
| | ERKAKEFQELLKELSERSEE |
| | LIRELEEKGAASEAELARMK |
| | QQHMTAYLEAQLTAWEIESK |
| | SKIALLELQQNQLNLELRHI |
| TMHC6 (SEQ ID NO: 1) | TENEIRKLRKLLRIAMLLLV |
| | FLLIATVVSLWTSKTDDDPS |
| | AQSEQLVAMSLMLIAASLLI |
| | IAISKLLKSRNG |
| TMHC8 (SEQ ID NO: 3) | SAEELLRRSREYLKKVALIQ |
| | LVIAFVFLILLILLSWRSEE |
| | LIRELEEKGAASEAELARMK |
| | QQHMTAYLQAALTAWEIISK |
| | SVIALLLLQQNQLNLELRHI |
| TMH4C4 (SEQ ID NO: 4) | SAEELLRRSREYLKKVALIQ |
| | LVIAFVFLILLILLSWRSEE |
| | LIRELEEKGAASEAELARMK |
| | QQHMTAYLQAALTAWEIISK |
| | SVIALLLLQQNQLNLELNTD |
| | TDKNVAEELLRRSREYLKKV |
| | ALIQLVIAFVFLILLILLSW |
| | RSEELIRELEEKGAASEAEL |
| | ARMKQQHMTAYLQAALTAWE |
| | IISKSVIALLLLQQNQLNLE |
| | LRH |

### 1.2 Design of Transmembrane Proteins

We designed transmembrane versions of WSHC6 and WSHC8 pores by resurfacing the outside of the crystal structures with patterned hydrophobic residues, and RK- and YW-rings at the intracellular and extracellular boundary region, respectively⁸. Briefly, the N- and C-termini of the transmembrane designs are designed to localize in the cytoplasmic side, by adding an RK ring with Arg and Lys residues close to the termini. YW ring, designed at the lipid-water boundary on the periplasmic side, set up the register in the membrane. We designed the hydrophobic transmembrane span in between the YW and RK rings with a length of 26 and 31 Å for TMHC6 and TMHC8, respectively. Hydrophobic residues are designed based on amino acid propensity in the membrane, replacing all polar residues exposed to the membrane. The crystal structure of WSHC8 was used as the starting model.

The residues surrounding the central pores were also redesigned to modulate compatibility within the membrane and channel conductance: if these residues are too hydrophobic, some lipids may block the pores; if too polar, the designed TM segment may not be able to insert into the membrane. Polar residues were designed in the TM pore region, while membrane compatibility was predicted by TMHMM³⁶ software. For TMHC6, we designed a ring of glutamate residues (E-ring) and two rings of lysine residues (K-rings) at the opening of the central channel on the extracellular and intracellular side, respectively, to modulate channel conductance. For TMHC8, in each monomer, we redesigned 4 charged residues surrounding the pore to apolar residues for insertion into the membrane, leaving 4 polar residues in the TM region to make a relative hydrophilic central channel. We ordered two designs for TMHC8, both of which expressed well, enriched in the membrane fraction. TMHMM³⁶ software predicts that both TMHC6 and TMHC8 have two transmembrane segments.

### 1.3 Figure Preparation for Structures and Maps and HOLE™ Calculations

All the figures for structures and maps are prepared in Chimera^{TM34} or Pymol™. The constriction sites in the channels and pores are calculated using HOLE²⁰. For the WSHC6 channel structure, the narrowest constriction is at Leu51 with a diameter of approximately 4 Å as calculated. For the TMHC6 design model, the narrowest constriction is at Glu44 with a diameter of approximately 3.3 Å. For the WSHC8 channel structure, the narrowest constriction of this channel has an inner diameter of approximately 10 Å at Lys80. For the TMH4C4 pore, the narrowest constriction is at Lys80 and Lys184 with a diameter of approximately 10 Å, calculated from EM structure.

### 2. Experimental Materials and Methods

### 2.1 Reagents

Chemicals used were of the highest grade commercially available and were purchased from Sigma-Aldrich (St. Louis, Mo., USA), Invitrogen (Carlsbad, Calif,, USA), or Qiagen (Hilden, Germany). Detergents were from Anatrace (Maumee, Ohio, USA).

### 2.2 Cloning and Expression of Water-Soluble Proteins

Synthetic genes were obtained from Genscript Inc. (Piscataway, N.J., USA) or IDT (Coralville, Iowa, USA) and cloned into the pET28b expression vector via NdeI/XhoI restriction sites. The plasmids were transformed into chemically competent E. coli Lemo21(DE3) cells (NEB, Ipswich, Mass.). Gene expression was facilitated by growing pre-cultures in Terrific Broth (TB) medium with a final concentration of 50 μg/ml kanamycin overnight at 37° C. 10 ml pre-cultures were used to inoculate 1 L of autoinduction medium³⁷, again containing 50 μg/ml kanamycin for plasmid selection. The cultures were grown at 37° C. for 3 hours and then 18° C. overnight for protein expression. Cells were harvested by centrifugation.

2.3 Cell lysis and Purification of Water-Soluble Proteins

Cells were resuspended and homogenized in lysis buffer containing 50 mM Tris-HCl pH 8.0 and 300 mM NaCl 20 mM imidazole, and lysed by sonication in the presence of DNAse and EDTA-free protease inhibitor tablet (ThermoFisher Scientific). Lysates were cleared by centrifugation at 4° C. 18,000 g for at least 30 minutes and applied to Ni-NTA (Qiagen) columns pre-equilibrated in the lysis buffer. The column was washed with 5-10 column volumes (CV) of wash buffer (50 mM Tris, 300 mM NaCl, 30 mM Imidazole, pH 8.0), followed by 5 CV of high-salt wash buffer (50 mM Tris, 1 M NaCl, 30 mM Imidazole, pH 8.0), and then 5 CV of high-imidazole wash buffer (50 mM Tris, 300 mM NaCl, 60 mM

Imidazole, pH 8.0). Protein was eluted with 50 mM Tris, 300 mM NaCl, 500 mM Imidazole, pH 8.0. After concentration, proteins were further purified by gel filtration (Superdex-200 increase 10/300 GL; GE Healthcare) using lx phosphate buffer saline (PBS), pH 7.4. The N-terminal hexa-histidine tag was removed with restriction grade thrombin (EMD Millipore) overnight at 30° C. After full cleavage, the reaction is stopped by addition of phenylmethanesulfonyl fluoride (PMSF), followed by another round of gel filtration purification.

### 2.4 Cloning and Expression of Transmembrane Proteins

Synthetic genes were obtained from Genscript Inc. (Piscataway, N.J., USA) or IDT (Coralville, Iowa, USA) and cloned into the pET29b expression vector via Ndel/Xhol restriction sites. The plasmids were transformed into chemically competent E. coli Lemo21(DE3) cells (NEB, Ipswich, Mass.). Gene expression was facilitated by growing pre-cultures in Luria-Bertani broth (LB) medium with a final concentration of 50 μg/ml kanamycin overnight at 37° C. 10 ml pre-cultures were used to inoculate 1 L of LB medium, again containing 50 μg/ml kanamycin for plasmid selection. The cultures were grown at 37° C. for 3-4 hours until an OD600 of 0.8-1.0 was reached and then 18° C. overnight for protein expression. Cells were harvested by centrifugation.

### 2.5 Cell Lysis and Purification of Transmembrane Proteins

Cells were resuspended and homogenized in lysis buffer containing 25 mM Tris-HCl pH 8.0 and 150 mM NaCl, and lysed by sonication in the presence of serine protease inhibitor phenylmethanesulfonyl fluoride (PMSF). Cell debris was removed by low-speed centrifugation for 10 minutes. The supernatant was collected and ultracentrifuged for 1 h at 150,000 g. The membrane fraction was collected and homogenized in buffer containing 25 mM Tris-HCl pH 8.0 and 150 mM NaCl. n-Decyl-β-D-Maltopyranoside (DM; Anatrace) was added to the membrane suspension to a final concentration of 1.5% (w/v) and then incubated for 2 h at 4° C. After another ultracentrifugation step at 150,000g for 30 min, the supernatant was collected and loaded on Ni²⁺-nitrilotriacetate affinity resin (Ni-NTA; Qiagen), followed by a wash with 25 mM Tris-HCl pH 8.0, 150 mM NaCl, 30 mM imidazole and 0.2% DM. Proteins were eluted with buffer containing 25 mM Tris-HCl pH 8.0, 150 mM NaCl, 30 mM imidazole and 0.2% DM. After concentration to 2-3 mg ml⁻¹, proteins were further purified by gel filtration (Superdex™-200 10/30; GE Healthcare). The buffer for gel filtration contained 25 mM Tris-HCl pH 8.0, 150 mM NaCl and 0.2% DM. To prepare samples for Cryo-EM, TMH4C4 protein was purified in the buffer containing 0.02% (weight/volume) n-dodecyl-β-D-maltopyranoside (DDM, Anatrace). The major peak fractions were taken for further Cryo-EM investigation. For AUC experiments, the proteins, originally in 0.2% DM buffer, were buffer exchanged into the buffer for AUC by gel filtration, which contained 20 mM sodium phosphate, pH 7.0 and 200 mM NaCl supplemented with 0.5% Pentaethylene Glycol Monooctyl Ether (C8E5).

### 2.6 Circular Dichroism (CD) Measurements

CD wavelength-scan measurements were made on an AVIV™ CD spectrometer model 420. Protein concentrations ranged from 0.1-0.2 mg/ml in PBS (pH 7.4) for soluble proteins and PBS (pH 7.4) plus 0.2% DM for transmembrane proteins. Wavelength-scan spectra from 260 to 190 nm were recorded in triplets and averaged. The scanning increment for full wavelength scans was 1 nm. Temperature melts were conducted in 2° C. steps (heating rate of 2° C./min) and recorded by following the absorption signal at a wavelength of 220 nm. Three sets of wavelength scan spectra were recorded at 25° C., 95° C. and after cooling down to 25° C.

### 2.7 Analytical Ultracentrifugation (AUC) Molecular Weight Determination

Analytical ultracentrifugation (sedimentation velocity and sedimentation equilibrium) experiments were carried out using a Beckman XL-I analytical ultracentrifuge (Beckman Coulter) equipped with an eight-cell An-50 Ti rotor. The protein samples were centrifuged at the speed of 40 k rpm for sedimentation velocity (SV) experiments. Sedimentation-equilibrium (SE) experiments were then performed on samples that showed monodisperse distribution (>90%) of sedimentation coefficients by SV to determine the MW of the discrete species. The water-soluble proteins were centrifuged in PBS (pH7.4). The transmembrane proteins were centrifuged in a buffer containing 20 mM sodium phosphate, pH 7.0, 200 mM NaCl and 0.5% C8E5. Density matching was unnecessary as the solvent density was equal to that of C8E5. For sedimentation equilibrium, data were collected by UV absorbance at 230 nm at 20° C. for three protein concentrations in the range of 0.2-0.8 OD23o. Samples were centrifuged at three different speeds corresponding to 6, the reduced molecular weight³⁸, of 2, 3 and 4.5 cm⁻², respectively. Data were globally fit to a single ideal species model using the program SEDPHAT. The partial specific volume, buffer density, and viscosity were calculated using SEDNTERP.

### 2.8 Small-Angle X-Ray Scattering

Samples were purified by gel filtration in PBS (pH7.4) with 1% glycerol; fractions preceding the void volume of the column were used as blanks for buffer subtraction. Scattering measurements were performed at the SIBYLS™ 12.3.1 beamline at the Advanced Light Source³⁹. For each sample, data were collected for two different concentrations to test for concentration-dependent effects; “high” concentration samples ranged from 4-7 mg/ml and “low” concentration samples ranged from 1-2 mg/ml. Data were analyzed using ATSAS™ software⁴⁰ and the experimental scattering profiles were fit to design models using CRYSOL^{TM41}.

2.9 X-Ray Crystallography

WSHC8 samples for crystallization were purified with an additional gel filtration step using a Superdex™ 200 Increase 10/300 GL column (GE Healthcare) with 20 mM Tris-HCl buffer pH8.0, 100 mN NaCl. Pure fractions were pooled and concentrated to 34 mg/mL. Crystallization screens were prepared using a 5-position deck Mosquito™ Crystal (ttplabtech) with an active humidity chamber. Crystals were grown by vapor diffusion in hanging drops at 17° C. Single crystals for data collection were grown in the No. 85 crystallization solution of JCSG+ suite (Qiagen) containing 0.3 M magnesium formate and 0.1 M Bis-Tris pH5.5. WSHC8 crystals were frozen in liquid nitrogen in the crystal growing buffer with additional 20% glycerol as cryo-protectant. The X-ray datasets were collected at the Advanced Light Source at Lawrence Berkeley National Laboratory with beamlines 8.2.1. Crystals are grown in the space group of P2₁2₁2. The structure of WSHC8 was determined using molecular replacement with the Rosetta™ design model. The model building was done with COOT^{TM45}. Structure refinement performed with PHENIX^{TM42}.

TABLE 3
Crystallographic statistics

| | WSHC8 | |
|---|---|---|
| Data collection | | |
| Space group | P2₁2₁2 | |
| Cell dimensions | | |
| a, b, c (Å) | 59.44, 103.68, 72.98 | |
| a, b, g (°) | 90, 90, 90 | |
| Resolution (Å) | 34.4-2.4 | (2.49-2.4) |
| R_{sym} or R_{merge} | 0.192 | (1.698) |
| I/sI | 8.3 | (1.4) |
| Completeness (%) | 99.6 | (99.4) |
| Redundancy | 7.0 | (7.2) |

| Refinement | |
|---|---|
| Resolution (Å) | 34.4-2.4 |
| No. reflections | 18183 |
| R_{work}/R_{free} | 0.261/0.298 |
| No. atoms | 2931 |
| Protein | 2914 |
| Ligand/ion | |
| Water | 17 |
| B-factors | 59.4 |
| Protein | 59.5 |
| Ligand/ion | |
| Water | 43.8 |
| R.m.s. deviations | |
| Bond lengths (Å) | 0.002 |
| Bond angles (°) | 0.37 |

* Data for WSHC8 structure were collected from a single crystal. Values in parentheses are for highest-resolution shell.
^{#} I/σ(I) (along a*, b* and c* axes)

### 2.10 Whole-Cell Patch-Clamp Experiments

Recombinant baculovirus was generated by using the Bac-to-Bac™ system (Invitrogen). Trichoplusia ni insect cells (HiS, Thermo Fisher) were grown on 35-mm Petri dishes in Grace's™ insect medium (Gibco) supplemented with FBS (10%) and antibiotics (100 μg/ml streptomycin and 100 U/ml penicillin). Cells were infected by replacing the incubation medium with a medium containing the baculovirus encoding the designed channels protein constructs. After 1 h, 2 ml incubation medium was added to the virus-containing medium. Cells were maintained at 25-27° C. for at least 24 h before the study.

Whole-cell patch clamp currents were recorded using an amplifier (Axopatch™ 200; Molecular Devices) with glass micropipettes resistance (1.5-3 MΩ). Capacitance was subtracted and series resistance was compensated using internal amplifier circuitry. To test the ion selectivity of the designed channels, Hi5 cells expressing the design of interest were bathed in a solution containing 100 mM of the chloride salt of the monovalent cations K⁺, Na⁺, Cs⁺, and CH₃NH₃⁺. The patch pipettes contained the equivalent concentration of the fluoride salt of the same cation, except for CH₃NH₃⁺, for which the pipette contained 100 mM CsF. The standard voltage-clamp protocol for measuring ionic currents consisted of 20-ms test pulses from a holding potential of 0 mV to voltages ranging from −100 mV to +100 mV in 10-mV steps. No leak subtraction protocols were used during the measuring of currents. The data were filtered at 2 KHz. For analysis of measured ionic current, the value of the current at the end of each pulse was measured. All current measurements were normalized to the cell capacitances. Each measurement is carried out on a different cell and the reported values are for the number of cells studied. The error bars in figures indicate the standard error of measurement (s.e.m.). Voltage-clamp pulses were generated, and currents were recorded using Pulse™ software controlling an Instrutech™ ITC18 interface (HEKA). Data was analyzed using Igor Pro 6.37 software (WaveMetrics).

### 2.11 MTS Blocking Experiments

The methanethiosulfonate (MTS) reagents [2-(trimethy-lammonium)ethyl] methanethiosulfonate (MTSET), N,N-Dibutyl-N-[2-(2-methyl-2,2-dioxidodithio)ethyl]-1-butanaminium Bromide; (MTS-TBAE) and sodium(2-sulfonatoethyl) methanethiosulfonate (MTSES) were purchased from Toronto Research Chemicals (North York, Canada). A 10 mM stock solution of MTS reagents dissolved in water was prepared, and aliquots of the stock solution were kept on ice until used. Before perfusing MTS reagents, the previously mentioned pulse protocol was applied to measure the peak current at +100 mV. Then, cells were perfused with 2.5 mM MTS reagent for 5-10 min while they are being held at 0 mV. This time was needed to allow the reagent to react with the SH groups of cysteine residues.

1. A polypeptide comprising an amino acid sequence at least 50% identical to the amino acid sequence of any one of SEQ ID NOS: 1-4.

2. (canceled)

3. The polypeptide of claim 1, wherein the polypeptide comprises an amino acid sequence at least 50% identical to the amino acid sequence of SEQ ID NO: 1, wherein the polypeptide is identical to the polypeptide of SEQ ID NO:1 at least at 1, 2, 3, 4, 5, 6, 7, 8, 9, 10, 11, 12, 13, 14, 15, 16, 17, 18, 19, 20, 21, 22, or more of residues 3, 13-14, 16-18, 20, 21, 23-25, 27-28, 30-32, 40, 44, 47, 49, 56, 65, and 68 in SEQ ID NO:1.

4. The polypeptide of claim 1, wherein the polypeptide comprises an amino acid sequence at least 50% identical to the amino acid sequence of SEQ ID NO: 1, wherein changes in the polypeptide amino acid sequence relative to SEQ ID NO:1 comprise:
(a) changes in 1, 2, 3, 4, 5, 6, 7, 8, 9, 10, 11, 12, 13, 14, 15, 16, 17, 18, 19, 20, 21, or more of residues 1-4, 6-7, 9-10, 32-40, 42, and 70-72 to other polar amino acids;
(b) changes in 1, 2, 3, 4, 5, 6, 7, 8, 9, 10, 11, 12, 13, or more of residues 14, 16-18, 20-21, 23-25, 27-28, 30, 49, 56, and 63 to other hydrophobic amino acids; and/or
(c) a change at residue 44.

5. The polypeptide of claim 1, wherein the polypeptide is identical to SEQ ID NO:1 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, or all of residues 5, 8, 11-12, 15, 19, 22, 26, 29, 41, 43, 45-48, 50, 51-55, 57-62, 64, and 67-69 in SEQ ID NO:1.

6. The polypeptide of claim 1, wherein the polypeptide is identical to SEQ ID NO:1 at 1, 2, or all 3 of residues 13, 31, and 65 in SEQ ID NO:1.

7. The polypeptide of claim 1 2, wherein the polypeptide comprises an amino acid sequence at least 50% identical to the amino acid sequence of SEQ ID NO: 2, wherein changes in the polypeptide amino acid sequence relative to SEQ ID NO:2 comprise changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14-15, 17-19, 21-26, 28-29, 32-33, 36-37, 39-40, 43-44, 46-55, 58, 60, 67, 71, 78, 82, 96, and 98-100 to other polar amino acids.

8. The polypeptide of claim 1, wherein the polypeptide is identical to SEQ ID NO:2 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:2.

9. The polypeptide of claim 1, wherein the polypeptide is identical to SEQ ID NO:2 at least at 1, 2, 3, 4, 5, 6, 7, 8, 9, or more of residues 62, 66, 69, 73, 76, 80, 83, 87, 91, and 94 in SEQ ID NO:2.

10. The polypeptide of claim 1, wherein the polypeptide comprises an amino acid sequence at least 50% identical to the amino acid sequence of SEQ ID NO: 3, wherein changes in the polypeptide amino acid sequence relative to SEQ ID NO:3 comprise (a) changes in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of residues 1-4, 7-8, 10-12, 14, 37, 39-40, 43-44, 46-55, 58, 60, 96, and 98-100 to other polar amino acids; and/or
(b) changes in 1, 2, 3, 4, 5, 6, 7, 8, 9, 10, 11, 12, 13, 14, 15, or more of residues 17-19, 21-26, 28-29, 32-33, 71, 78, and 82 to other hydrophobic amino acids.

11. The polypeptide of claim 1 wherein the polypeptide is identical to SEQ ID NO:3 at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or all of residues 5-6, 9, 13, 16, 20, 27, 30-31, 34-35, 38, 41-42, 45, 56-57, 59, 61, 63-65, 68, 70, 72, 74-75, 77, 79, 81, 84-86, 88-90, 92-93, 95, and 97 in SEQ ID NO:3.

12. The polypeptide of claim 1, wherein the polypeptide is identical to SEQ ID NO:3 at 1, 2, or all 3 of residues 15, 36, and 66 in SEQ ID NO:3.

13. The polypeptide of claim 1, wherein the polypeptide is identical to SEQ ID NO:3 at least at 1, 2, 3, 4, 5, 6, 7, 8, 9, or more of residues 62, 66, 69, 73, 76, 80, 83, 87, 91, and 94 in SEQ ID NO:3.

14. (canceled)

15. The polypeptide of claim 1, wherein the polypeptide is of the formula X1-Z—X2, wherein X1 and X2 independently comprise a polypeptide of claim 1, and Z comprises an amino acid linker.

16. (canceled)

17. The polypeptide of claim 15, wherein the polypeptide comprises an amino acid sequence at least 50% identical to the amino acid sequence of SEQ ID NO:4.

18. The polypeptide of claim 15, wherein the amino acid linker comprises a flexible amino acid linker at least 4 amino acid residues in length.

19. A polypeptide oligomer comprising 2, 3, 4, 5, 6, or more copies of claim 1.

20. (canceled)

21. A nucleic acid encoding the polypeptide of claim 1.

22. An expression vector comprising the nucleic acid of claim 21 operatively linked to a suitable control sequence.

23. A host cell comprising the expression vector of claim 23.

24. (canceled)

25. A liposome comprising the polypeptide oligomer of claim 19, wherein the polypeptide oligomer forms a transmembrane channel in the liposome.

26. (canceled)
